(19) United States
(12) Patent Application Publication
(10) Pub. No.: US 2022/0034885 A1
(43) Pub. Date: Feb. 3, 2022

(54) COMPOSITIONS AND METHODS FOR DETERMINING CORONAVIRUS NEUTRALIZATION TITERS

(71) Applicant: ADMA Biologics, Inc., Ramsey, NJ (US)

(72) Inventors: James Mond, Silver Spring, MD (US); William Pat Leinert, SR., Fenton, MO (US); Andy Gibson, Fenton, MO (US)

(21) Appl. No.: 17/391,990

(22) Filed: Aug. 2, 2021

Related U.S. Application Data
(60) Provisional application No. 63/060,395, filed on Aug. 3, 2020.
(60) Provisional application No. 63/074,892, filed on Sep. 4, 2020.

(51) Int. Cl. G01N 33/569; G01N 33/58; G01N 33/68

(52) U.S. Cl. CPC G01N 33/56983; G01N 33/581; G01N 33/6854; G01N 2800/12; G01N 2333/165; G01N 2333/908; G01N 2500/02; G01N 2469/20

(57) ABSTRACT

The disclosure is directed to methods and kits for detecting neutralizing antibodies against a coronavirus (e.g., SARS-CoV-2) in a sample, such as a plasma sample or pooled plasma composition. The methods utilize a panel of SARS-CoV-2 neutralizing antibodies as a positive control. The kit may be a rapid detection kit that measures neutralizing antibodies using the provided methods.

### CROSS REFERENCE TO RELATED APPLICATIONS

This application claims priority to and the benefit of U.S. Provisional Patent Application No. 63/060,395 filed Aug. 3, 2020, and U.S. Provisional Patent Application No. 63/074,892 filed Sep. 4, 2020, both of which are incorporated herein by reference in their entireties for all purposes.

### FIELD

The present disclosure relates to methods and compositions for detecting coronavirus neutralizing antibodies in a sample, such as a plasma sample or a pooled plasma composition. Compositions and methods of the disclosure find use in, among other things, clinical, therapeutic and preventative medicine and research applications.

### BACKGROUND

According to the U.S. Department of Health and Human Services Centers for Disease Control and Prevention (CDC), Chinese authorities identified an outbreak caused by a novel coronavirus termed SARS-CoV-2. The virus can cause mild to severe respiratory illness, known as Coronavirus Disease 2019 (COVID-19), formerly called 2019-nCoV (van Dorp L et al., Infec Genet Evol, 2020; 83:104351). The outbreak began in Wuhan, Hubei Province, China and has spread to a growing number of countries worldwide, including the United States. On Mar. 11, 2020, the World Health Organization declared COVID-19 a pandemic. SARS-CoV-2 is different from six other, previously identified human coronaviruses, including those that have caused previous outbreaks of Severe Acute Respiratory Syndrome (SARS) and Middle East Respiratory Syndrome (MERS).

The U.S. Food and Drug Administration (FDA) has not yet approved a drug specifically indicated for the treatment of COVID-19. As such, plasma from patients who have recovered from COVID-19 (also known as “convalescent plasma”) is being explored as a possible therapeutic for severe cases of COVID-19. Thus, there remains a need for methods and compositions to identify plasma samples that have titers of coronavirus neutralizing antibodies.

### SUMMARY

The present disclosure provides a method of detecting coronavirus neutralizing antibodies in a sample, which method comprises: (a) contacting a sample with a solid support comprising a coronavirus cell receptor or portion thereof (e.g., capable of binding to a coronavirus or portion thereof) immobilized thereto to form a mixture; (b) contacting the mixture with a conjugate comprising a reporter molecule attached to a coronavirus attachment moiety or peptide (e.g., a peptide comprising a receptor binding domain (RBD) of a coronavirus spike protein), whereby, if coronavirus neutralizing antibodies are present in the sample, the coronavirus attachment moiety (e.g., RBD) binds to the coronavirus neutralizing antibodies and does not bind to the immobilized coronavirus cell receptor or portion thereof; (c) detecting and quantifying a signal from the reporter molecule, wherein the amount of detected signal is inversely proportional to the amount of coronavirus neutralizing antibodies present in the sample; and (d) performing steps (a)-(c) on a positive control comprising a panel of one or more coronavirus neutralizing monoclonal antibodies instead of the sample, and determining the neutralizing antibody capacity present in the sample (e.g., via comparing the quantified signal of the positive control to the quantified signal of the sample). The method further provides determining the neutralizing antibody titer present in the sample based on the positive control, by correlating the neutralizing antibody capacity of the positive control to its neutralizing antibody titer (e.g., as determined by a plaque reduction neutralization test (PRNT) or a focus reduction neutralization test (FRNT)).

The present disclosure also provides a method of detecting coronavirus neutralizing antibodies in a sample, which method comprises: (a) contacting a sample with a conjugate comprising a reporter molecule attached to a coronavirus attachment moiety or peptide (e.g., a peptide comprising a receptor binding domain (RBD) of a coronavirus spike protein) to form a mixture, wherein the coronavirus attachment moiety (e.g., RBD) binds to coronavirus neutralizing antibodies if present in the sample; (b) contacting the mixture with a solid support comprising a coronavirus cell receptor or portion thereof (e.g., capable of binding to a coronavirus or portion thereof) immobilized thereto, whereby, if coronavirus neutralizing antibodies are present in the sample, the coronavirus attachment moiety (e.g., RBD) bound to the coronavirus neutralizing antibodies does not bind to the immobilized coronavirus cell receptor or portion thereof; (c) detecting and quantifying a signal from the reporter molecule, wherein the amount of detected signal is inversely proportional to the amount of coronavirus neutralizing antibodies present in the sample; and (d) performing steps (a)-(c) on a positive control comprising a panel of one or more coronavirus neutralizing monoclonal antibodies instead of the sample, and comparing the quantified signal of the positive control to the quantified signal of the sample to determine the neutralizing antibody capacity present in the sample. The method further provides determining the neutralizing antibody titer present in the sample based on the positive control, by correlating the neutralizing antibody capacity of the positive control to its neutralizing antibody titer (e.g., as determined by a plaque reduction neutralization test (PRNT) or a focus reduction neutralization test (FRNT)).

The disclosure further provides a method of identifying plasma comprising coronavirus neutralizing antibodies, which method comprises (a) contacting a plasma sample with a solid support comprising a coronavirus cell receptor or portion thereof immobilized thereto to form a mixture; (b) contacting the mixture with a conjugate comprising a reporter molecule attached to a coronavirus attachment moiety or peptide (e.g., a peptide comprising a receptor binding domain (RBD) of a coronavirus spike protein), whereby, if coronavirus neutralizing antibodies are present in the plasma sample, the coronavirus attachment moiety (e.g., RBD peptide) binds to the coronavirus neutralizing antibodies and does not bind to the immobilized coronavirus cell receptor; (c) detecting and quantifying a signal from the reporter molecule, wherein the amount of detected signal is inversely proportional to the amount of coronavirus neutralizing antibodies present in the plasma sample; and (d) performing steps (a)-(c) on a positive control comprising a panel of one or more coronavirus neutralizing monoclonal antibodies instead of the plasma sample, and comparing the quantified signal of the positive control to the quantified signal of the plasma sample to determine the neutralizing antibody capacity present in the sample. The method further provides determining the neutralizing antibody titer present in the sample based on the positive control, by correlating the neutralizing antibody capacity of the positive control to its neutralizing antibody titer (e.g., as determined by a plaque reduction neutralization test (PRNT) or a focus reduction neutralization test (FRNT)).

In other aspects, the present disclosure provides a method of identifying plasma comprising coronavirus neutralizing antibodies which method comprises: (a) contacting a plasma sample with a conjugate comprising a reporter molecule attached to a coronavirus attachment moiety or peptide (e.g., a peptide comprising a receptor binding domain (RBD) of a coronavirus spike protein) to form a mixture, wherein the coronavirus attachment moiety (e.g., RBD peptide) binds to coronavirus neutralizing antibodies if present in the plasma sample; (b) contacting the mixture with a solid support comprising a coronavirus cell receptor or portion thereof immobilized thereto, whereby, if coronavirus neutralizing antibodies are present in the plasma sample, the coronavirus attachment moiety (e.g., RBD peptide) bound to the coronavirus neutralizing antibodies does not bind to the immobilized coronavirus cell receptor; (c) detecting and quantifying a signal from the reporter molecule, wherein the amount of detected signal is inversely proportional to the amount of coronavirus neutralizing antibodies present in the plasma sample; and (d) performing steps (a)-(c) on a positive control comprising a panel of one or more coronavirus neutralizing monoclonal antibodies instead of the plasma sample, and comparing the quantified signal of the positive control to the quantified signal of the plasma sample to determine the neutralizing antibody capacity present in the sample. The method further provides determining the neutralizing antibody titer present in the sample based on the positive control, by correlating the neutralizing antibody capacity of the positive control to its neutralizing antibody titer (e.g., as determined by a plaque reduction neutralization test (PRNT) or a focus reduction neutralization test (FRNT)).

Also provided is a method of producing an immune globulin comprising elevated levels of neutralizing antibody titers to one or more coronaviruses, which comprises (a) pooling plasma samples from a plurality of human plasma donors to produce a pooled plasma composition, and (b) detecting coronavirus neutralizing antibodies in the pooled plasma composition using any one or more of the methods disclosed herein. In some aspects, utilizing the one or more methods disclosed herein, it is possible to generate a pooled plasma composition and/or immunoglobulin prepared therefrom having a standardized coronavirus neutralizing antibody capacity and/or titer. The disclosure is not limited to any particular standardized coronavirus neutralizing antibody capacity and/or titer. Indeed, any standardized coronavirus neutralizing antibody capacity and/or titer that is useful for the treatment and/or prevention of infection by coronavirus may be generated. In some aspects, the coronavirus neutralizing antibody titer in the pooled plasma composition is at least 300.

The present disclosure further provides a rapid detection kit for detecting coronavirus neutralizing antibodies, which comprises: (a) a solid support comprising a coronavirus cell receptor or portion thereof immobilized thereto; (b) a conjugate comprising a reporter molecule attached to a coronavirus attachment moiety or peptide (e.g., a peptide comprising a receptor binding domain (RBD) of a coronavirus spike protein); (c) a positive control comprising panel of one or more coronavirus neutralizing antibodies; and (d) a negative control comprising at least one coronavirus non-neutralizing antibody.

### BRIEF DESCRIPTION OF THE DRAWING(S)

FIG. 1 is a dose dependent binding curve of a monoclonal antibody specific to recombinant SARS-CoV-2 receptor binding domain (RBD) protein. This curve is the basis for the serology assay for detection of SARS-CoV-2 IgG antibodies described in Example 2 and the ranking of plasma samples.

FIG. 2A is a dose dependent binding curve of the SARS-CoV-2-receptor binding domain (RBD) conjugated to horseradish peroxidase (HRP) titered against immobilized ACE2 in wells of a polystyrene micro-titer plate. The signal measured was absorbance OD₄₅₀ using TMB as a chromogenic substrate. FIG. 2B is a graph showing test results on plasma samples that tested positive (+, ++, +++, ++++), negative (−), and borderline (+/−) for RBD antibodies using a separate RBD antigen ELISA serology test. FIG. 2B also shows various levels of neutralizing activity of these plasma samples using the assay's algorithm for calculating % neutralization. Plasma samples with a ++ titer for RBD antibodies by serology can have varying degrees of neutralizing activity as demonstrated by the algorithm in this neutralization assay. FIG. 2C is a graph showing test results using an assay algorithm to discriminate various levels of neutralizing activity for four separate monoclonal antibodies known to neutralize the SARS-CoV-2 virus in vivo and in vitro.

FIG. 3A is a graph showing results of a high-throughput screening for SARS-CoV-2 neutralizing antibodies using a COVID-19 neutralization ELISA assay described herein. Clones A, B, C and D recognize the r-SARS-CoV-2 receptor binding domain (RBD) and clone E is specific for the r-N-terminal domain (NTD) of the spike protein. Antibodies were spiked in negative human plasma collected in 2020. FIG. 3B is a graph of dose-dependent binding curves from a panel of monoclonal antibodies isolated from COVID-19 survivors using the COVID-19 micro-ELISA serology assay. Clones A, B, C and D recognize the r-SARS-CoV-2 receptor binding domain (RBD) and clone E is specific for the r-N-terminal domain (NTD) of the spike protein.

FIG. 4 is a graph showing a comparison between a COVID-19 neutralization micro-ELISA assay as described herein (“ImmunoRank” in FIG. 4) and a live virus focus reduction neutralization test (FRNT50).

FIG. 5 is a graph showing that the % coronavirus neutralization (SNI) is independent of amount of binding (SCR) detected (see Table 6). For example, data points in the upper left of the graph (above the trendline) are samples that have high neutralization (50%-60%) but low binding, and data points below the trendline are samples with high binding (>2) but low % neutralization (>30%).

FIG. 6 is a graph showing the % coronavirus neutralization (SNI) values for various samples as a function of density. A value designated as a “low positive” was assigned a range between 0.2 and 0.4; a value designated as a “medium positive” was assigned a range between 0.4 and 0.7, and a value designated as a “high positive” was assigned a range between 0.7 and 1.

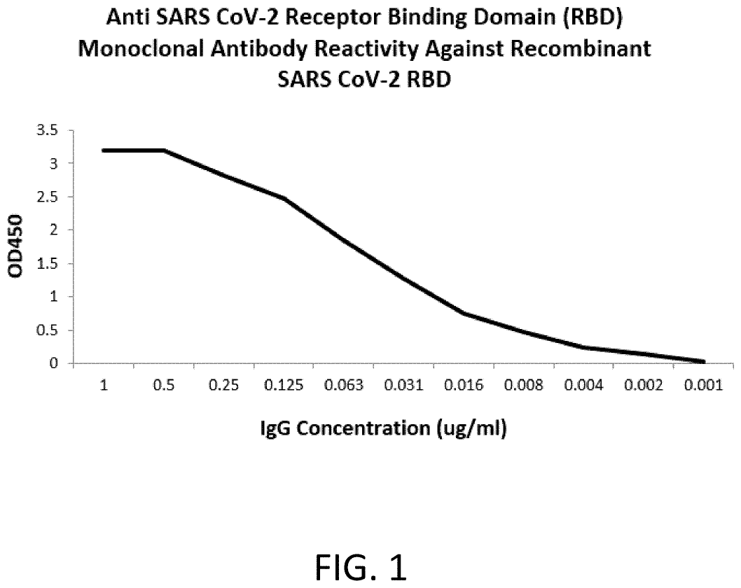

### DETAILED DESCRIPTION

The present disclosure is predicated, at least in part, on the development of a COVID-19 neutralization assay that rapidly detects neutralizing antibodies in immunoglobulin, plasma, serum, other blood solutions, or cell culture fluid that bind to a coronavirus attachment moiety or peptide (e.g., a peptide comprising a receptor binding domain (RBD) of a coronavirus spike protein) of SARS-CoV-2 and are capable of blocking the binding of the coronavirus attachment moiety or peptide to the coronavirus cell receptor or portion thereof (e.g., including, but not limited to, angiotensin-converting enzyme 2 (ACE2)). For example, by blocking the binding of the RBD to ACE2, the virus is unable to use its primary mode of entry into target cells to further its infection. Neutralization of the virus, rather than merely antibody binding to the virus, has been shown to correlate with clinical efficacy. Thus, the methods described herein may be used in various settings including, but not limited to, identifying subjects harboring a desired coronavirus neutralizing antibody capacity and/or titer, to assess whether a particular vaccine induces a sufficient neutralizing antibody response to mediate clinical protection, and to screen plasma for its ability to provide protection from the coronavirus or to mediate a therapeutic effect.

### Definitions

To facilitate an understanding of the present technology, a number of terms and phrases are defined below. Additional definitions are set forth throughout the detailed description.

As used herein, the term “subject” refers to any human or animal (e.g., non-human primate, rodent, feline, canine, bovine, porcine, equine, etc.).

As used herein, the term “sample” is used in its broadest sense and encompass materials obtained from any source. As used herein, the term “sample” is used to refer to materials obtained from a biological source, for example, obtained from animals (including humans), and encompasses any fluids, solids and tissues. In particular embodiments of the present disclosure, biological samples include blood and blood products such as plasma, serum and the like. However, these examples are not to be construed as limiting the types of samples that find use with the present disclosure.

As used herein, the term “antibody” refers to an immunoglobulin molecule that is typically composed of two identical pairs of polypeptide chains, each pair having one “light” (L) chain and one “heavy” (H) chain. Human light chains are classified as kappa and lambda light chains. Heavy chains are classified as mu, delta, gamma, alpha, or epsilon, and define the antibody's isotype as IgM, IgD, IgG, IgA, and IgE, respectively. Within light and heavy chains, the variable and constant regions are joined by a “J” region of about 12 or more amino acids, with the heavy chain also including a “D” region of about 3 or more amino acids. Each heavy chain is comprised of a heavy chain variable region (abbreviated herein as HCVR or V_{H}) and a heavy chain constant region. The heavy chain constant region is comprised of three domains, C_{H1}, C_{H2} and C_{H3}. Each light chain is comprised of a light chain variable region (abbreviated herein as LCVR or V_{L}) and a light chain constant region. The light chain constant region is comprised of one domain, CL. The constant regions of the antibodies may mediate the binding of the immunoglobulin to host tissues or factors, including various cells of the immune system (e.g., effector cells) and the first component (C1q) of the classical complement system. The V_{H} and V_{L} regions can be further subdivided into regions of hypervariability, termed complementarity determining regions (CDR), interspersed with regions that are more conserved, termed framework regions (FR). Each V_{H} and V_{L} is composed of three CDRs and four FRs, arranged from amino-terminus to carboxy-terminus in the following order: FR1, CDR1, FR2, CDR2, FR3, CDR3, FR4. The variable regions of each heavy/light chain pair (V_{H} and V_{L}), respectively, form the antibody binding site. The term “antibody” encompasses an antibody that is part of an antibody multimer (a multimeric form of antibodies), such as dimers, trimers, or higher-order multimers of monomeric antibodies. It also encompasses an antibody that is linked or attached to, or otherwise physically or functionally associated with, a non-antibody moiety. Further, the term “antibody” is not limited by any particular method of producing the antibody. For example, it includes, inter alia, recombinant antibodies, synthetic antibodies, monoclonal antibodies, polyclonal antibodies, bi-specific antibodies, and multi-specific antibodies.

As used herein, the term “antibody derivative” or “derivative” of an antibody refers to a molecule that is capable of binding to the same antigen that the antibody from which it is derived binds to and comprises an amino acid sequence that is the same or similar to the antibody linked to an additional molecular entity. The amino acid sequence of the antibody that is contained in the antibody derivative may be the full-length antibody, or may be any portion or portions of a full-length antibody. The additional molecular entity may be a chemical or biological molecule. Examples of additional molecular entities include chemical groups, amino acids, peptides, proteins (such as enzymes, antibodies), and chemical compounds. The additional molecular entity may have any utility, such as for use as a detection agent, label, marker, pharmaceutical or therapeutic agent. The amino acid sequence of an antibody may be attached or linked to the additional entity by chemical coupling, genetic fusion, noncovalent association or otherwise. The term “antibody derivative” also encompasses chimeric antibodies, humanized antibodies, and molecules that are derived from modifications of the amino acid sequences of an antibody, such as conservation amino acid substitutions, additions, and insertions.

As used herein, the term “antigen” refers to any substance that is capable of inducing an immune response. An antigen may be whole cell (e.g. bacterial cell), virus, fungus, or an antigenic portion or component thereof. Examples of antigens include, but are not limited to, microbial pathogens, bacteria, viruses, proteins, glycoproteins, lipoproteins, peptides, glycopeptides, lipopeptides, toxoids, carbohydrates, tumor-specific antigens, and antigenic portions or components thereof.

As used herein, the term “antigen-binding fragment” of an antibody refers to one or more portions of a full-length antibody that retain the ability to bind to the same antigen that the antibody binds to.

The terms “specific binding partner,” “specific binding member,” and “binding member” are used interchangeably herein and refer to one of two or more different molecules that specifically recognize the other molecule compared to substantially less recognition of other molecules.

As used herein, when an antibody or other entity (e.g., antigen binding domain) “specifically recognizes” or “specifically binds” an antigen or epitope, it preferentially recognizes the antigen in a complex mixture of proteins and/or macromolecules, and binds the antigen or epitope with affinity which is substantially higher than to other entities not displaying the antigen or epitope. In this regard, “affinity which is substantially higher” means affinity that is high enough to enable detection of an antigen or epitope which is distinguished from entities using a desired assay or measurement apparatus. Typically, it means binding affinity having a binding constant (Kₐ) of at least 10⁷ M⁻¹ (e.g., >10⁷ M⁻¹, >10⁸ M⁻¹, >10⁹ M⁻¹, >10¹⁰ M⁻¹, >10¹¹ M⁻¹, >10¹² M⁻¹, >10¹³ M⁻¹, etc.). In certain such embodiments, an antibody is capable of binding different antigens so long as the different antigens comprise that particular epitope. In certain instances, for example, homologous proteins from different species may comprise the same epitope.

As used herein, the terms “immune globulin,” “immunoglobulin,” “immunoglobulin molecule” and “IG” encompass (1) antibodies, (2) antigen-binding fragments of an antibody, and (3) derivatives of an antibody, each as defined herein. As described herein, immunoglobulin may be prepared from (e.g., fractionated from, isolated from, purified from, concentrated from, etc.) pooled plasma compositions (e.g., for administration to a subject). As used herein, the term intravenous immune globulin (IVIG) and the like, for example, coronavirus-IVIG, refers to immune globulin prepared from a plurality of human donors that contains an elevated coronavirus specific antibody titer compared to a control sample (e.g., conventional IVIG prepared from a mixture of plasma samples obtained from 100 or more random human plasma donors).

As used herein, the term “antibody sample” refers to an antibody-containing composition (e.g., fluid (e.g., plasma, blood, purified antibodies, blood or plasma fractions, blood or plasma components etc.)) taken from or provided by a donor (e.g., natural source) or obtained from a synthetic, recombinant, other in vitro source, or from a commercial source. The antibody sample may exhibit elevated titer of a particular antibody or set of antibodies based on the pathogenic/antigenic exposures (e.g., natural exposure or through vaccination) of the donor or the antibodies engineered to be produced in the synthetic, recombinant, or in vitro context. Herein, an antibody sample with elevated titer of antibody X is referred to as an “X-elevated antibody sample.” For example, an antibody sample with elevated titer of antibodies against cytomegalovirus is referred to as a “cytomegalovirus-elevated antibody sample.”

As used herein, the term “isolated antibody” or “isolated binding molecule” refers to an antibody or binding molecule that is identified and separated from at least one contaminant with which it is ordinarily associated in its source. Examples of an isolated antibody include: an antibody that: (1) is not associated with one or more naturally associated components that accompany it in its natural state; (2) is substantially free of other proteins from its origin source; or (3) is expressed recombinantly, in vitro, or cell-free, or is produced synthetically and the is removed the environment in which it was produced.

As used herein, the terms “pooled plasma,” “pooled plasma samples” and “pooled plasma composition” refer to a mixture of two or more plasma samples and/or a composition prepared from same (e.g., immunoglobulin). Elevated titer of a particular antibody or set of antibodies in pooled plasma reflects the elevated titers of the antibody samples that make up the pooled plasma. For example, plasma samples may be obtained from subjects that have been vaccinated (e.g., with a vaccine) or that have naturally high titers of antibodies to one or more pathogens as compared to the antibody level(s) found in the population as a whole. Upon pooling of the plasma samples, a pooled plasma composition is produced (e.g., that has elevated titer of antibodies specific to a particular pathogen). Herein, a pooled plasma with elevated titer of antibody X (e.g., wherein “X” is a microbial pathogen) is referred to as “X-elevated antibody pool.” For example, a pooled plasma with elevated titer of antibodies against cytomegalovirus is referred to as “cytomegalovirus-elevated antibody pool.” Also used herein is the term “primary antibody pool” which refers to a mixture of two or more plasma samples. Elevated titer of a particular antibody or set of antibodies in a primary antibody pool reflects the elevated titers of the antibody samples that make up the primary antibody pool. Pooled plasma compositions can be used to prepare immunoglobulin (e.g., that is subsequently administered to a subject) via methods known in the art (e.g., fractionation, purification, isolation, etc.). The present disclosure provides that both pooled plasma compositions and immunoglobulin prepared from same may be administered to a subject to provide prophylactic and/or therapeutic benefits to the subject. Accordingly, the term pooled plasma composition may refer to immunoglobulin prepared from pooled plasma/pooled plasma samples.

As used herein, the term “isolated antibody” or “isolated binding molecule” refers to an antibody or binding molecule that is identified and separated from at least one contaminant with which it is ordinarily associated in its source. Examples of an isolated antibody include: an antibody that: (1) is not associated with one or more naturally associated components that accompany it in its natural state; (2) is substantially free of other proteins from its origin source; or (3) is expressed recombinantly, in vitro, or cell-free, or is produced synthetically and the is removed the environment in which it was produced.

As used herein, the term “purified” or “to purify” means the result of any process that removes some of a contaminant from the component of interest, such as a protein (e.g., antibody) or nucleic acid. The percent of a purified component is thereby increased in the sample.

As used herein, the term “donor” refers to a subject that provides a biological sample (e.g., blood, plasma, etc.). A donor/donor sample may be screened for the presence or absence of specific pathogens (e.g., using U.S. Food and Drug Administration (FDA) guidelines for assessing safety standards for blood products (e.g., issued by the FDA Blood Products Advisory Committee). For example, a donor/donor sample may be screened according to FDA guidelines to verify the absence of one or more bloodborne pathogens (e.g., human immunodeficiency virus (HIV) 1 (HIV-1), HIV-2; Treponema pallidum (syphilis); Plasmodium falciparum, P. malariae, P. ovale, P. vivax or P. knowlesi (malaria); hepatitis B virus (HBV), hepatitis C virus HCV); prions (Creutzfeldt Jakob disease); West Nile virus; parvovirus; Typanosoma cruzi; coronavirus (e.g., coronavirus OC43, coronavirus 229E, coronavirus NL63, coronavirus HKU1, MERS-CoV, SARS-CoV, or SARS-CoV-2 (COVID-19)); vaccinia virus or other pathogen routinely screened or that is recommended to be screed for by a regulatory body such as the FDA). As used herein, the terms “selected donor,” “selected human subject” and the like refer to a subject that is chosen and/or identified to provide a biological sample (e.g., blood, plasma, etc.) based on the presence of a desired characteristic of that biological sample (e.g., a specific titer (e.g., high, average or low titer) of antibodies (e.g., determined using one or more screening methods (e.g., neutralization assay or other assay described herein) specific for one or more pathogens (e.g., one or more respiratory pathogens (e.g., respiratory syncytial virus))). As used herein, the terms a “non-selected donor,” “random donor,” “random human subject” and the like, when used in reference to a donor sample (e.g., blood, plasma, etc.) used for generating a pool of donor samples), refer to a subject that provides a biological sample (e.g., blood, plasma, etc.) without specific knowledge of characteristics (e.g., antibody titer to one or more pathogens) of that sample. Thus, a random donor/random donor sample may be a subject/sample that passes FDA bloodborne pathogen screening requirements and is not selected on the basis of antibody titers (e.g., respiratory pathogen specific antibody titers). In one embodiment described herein, the titer for non-tested/non-selected source donor/donor sample is set at zero. If biological samples from a group of selected donors selected for the same characteristic are pooled, the pool so generated (e.g., a primary pool) will be enhanced for the selected characteristic. On the other hand, if biological samples from a group of non-selected, random donors are pooled, random differences between the biological samples will be averaged out, and the pool so generated (e.g., the primary pool) will not be enhanced for any specific characteristic. It is preferred that both random donors/random donor samples and selected donors/selected donor samples are screened (e.g., using FDA screening requirements) to verify the absence of bloodborne pathogens (e.g., prior to and/or after pooling). Furthermore, according to one embodiment of the present disclosure, and as described in detail herein, biological samples (e.g., plasma samples) from one or more selected donors can be mixed with biological samples (e.g., plasma samples) from one or more other selected donors (e.g., selected for the same or different characteristic (e.g., the same or different titer (e.g., high, medium or low titer) of antibodies to a specific pathogen) and/or mixed with biological samples (e.g., plasma samples) from one or more non-selected donors in order to generate a pooled plasma composition (e.g., that contains a desired, standardized level of antibodies for one or more specific pathogens (e.g., one or more respiratory pathogens)).

The terms “buffer” or “buffering agents” refer to materials, that when added to a solution, cause the solution to resist changes in pH.

The term “solution” refers to an aqueous or non-aqueous mixture.

As used herein, the term “an amount effective to induce an immune response” (e.g., of a composition for inducing an immune response), refers to the dosage level required (e.g., when administered to a subject) to stimulate, generate and/or elicit an immune response in the subject. An effective amount can be administered in one or more administrations (e.g., via the same or different route), applications or dosages and is not intended to be limited to a particular formulation or administration route.

As used herein, the term “under conditions such that said subject generates an immune response” refers to any qualitative or quantitative induction, generation, and/or stimulation of an immune response (e.g., innate or acquired).

As used herein, the term “immune response” refers to a response by the immune system of a subject. For example, immune responses include, but are not limited to, a detectable alteration (e.g., increase) in Toll-like receptor (TLR) activation, lymphokine (e.g., cytokine (e.g., Th1 or Th2 type cytokines) or chemokine) expression and/or secretion, macrophage activation, dendritic cell activation, T cell activation (e.g., CD4+ or CD8+ T cells), NK cell activation, and/or B cell activation (e.g., antibody generation and/or secretion). Additional examples of immune responses include binding of an immunogen (e.g., antigen (e.g., immunogenic polypeptide)) to an MHC molecule and inducing a cytotoxic T lymphocyte (“CTL”) response, inducing a B cell response (e.g., antibody production), and/or T-helper lymphocyte response, and/or a delayed type hypersensitivity (DTH) response against the antigen from which the immunogenic polypeptide is derived, expansion (e.g., growth of a population of cells) of cells of the immune system (e.g., T cells, B cells (e.g., of any stage of development (e.g., plasma cells), and increased processing and presentation of antigen by antigen presenting cells. An immune response may be to immunogens that the subject's immune system recognizes as foreign (e.g., non-self antigens from microorganisms (e.g., pathogens), or self-antigens recognized as foreign). Thus, it is to be understood that, as used herein, “immune response” refers to any type of immune response, including, but not limited to, innate immune responses (e.g., activation of Toll receptor signaling cascade) cell-mediated immune responses (e.g., responses mediated by T cells (e.g., antigen-specific T cells) and non-specific cells of the immune system) and humoral immune responses (e.g., responses mediated by B cells (e.g., via generation and secretion of antibodies into the plasma, lymph, and/or tissue fluids). The term “immune response” is meant to encompass all aspects of the capability of a subject's immune system to respond to antigens and/or immunogens (e.g., both the initial response to an immunogen (e.g., a pathogen) as well as acquired (e.g., memory) responses that are a result of an adaptive immune response).

The term “recombinant,” as used herein, means that a particular nucleic acid (DNA or RNA) is the product of various combinations of cloning, restriction, polymerase chain reaction (PCR) and/or ligation steps resulting in a construct having a structural coding or non-coding sequence distinguishable from endogenous nucleic acids found in natural systems. DNA sequences encoding polypeptides can be assembled from cDNA fragments or from a series of synthetic oligonucleotides to provide a synthetic nucleic acid which is capable of being expressed from a recombinant transcriptional unit contained in a cell or in a cell-free transcription and translation system. Genomic DNA comprising the relevant sequences can also be used in the formation of a recombinant gene or transcriptional unit. Sequences of non-translated DNA may be present 5′ or 3′ from the open reading frame, where such sequences do not interfere with manipulation or expression of the coding regions, and may act to modulate production of a desired product by various mechanisms. Alternatively, DNA sequences encoding RNA that is not translated may also be considered recombinant. Thus, the term “recombinant” nucleic acid also refers to a nucleic acid which is not naturally occurring, e.g., is made by the artificial combination of two otherwise separated segments of sequence through human intervention. This artificial combination is often accomplished by either chemical synthesis means, or by the artificial manipulation of isolated segments of nucleic acids, e.g., by genetic engineering techniques. Such is usually done to replace a codon with a codon encoding the same amino acid, a conservative amino acid, or a non-conservative amino acid.

Alternatively, the artificial combination may be performed to join together nucleic acid segments of desired functions to generate a desired combination of functions. This artificial combination is often accomplished by either chemical synthesis means, or by the artificial manipulation of isolated segments of nucleic acids, e.g., by genetic engineering techniques. When a recombinant polynucleotide encodes a polypeptide, the sequence of the encoded polypeptide can be naturally occurring (“wild type”) or can be a variant (e.g., a mutant) of the naturally occurring sequence. Thus, the term “recombinant” polypeptide does not necessarily refer to a polypeptide whose sequence does not naturally occur. Instead, a “recombinant” polypeptide is encoded by a recombinant DNA sequence, but the sequence of the polypeptide can be naturally occurring (“wild type”) or non-naturally occurring (e.g., a variant, a mutant, etc.). Thus, a “recombinant” polypeptide is the result of human intervention, but may comprise a naturally occurring amino acid sequence.

As used herein, the term “pathogen product” refers to any component or product derived from a pathogen including, but not limited to, polypeptides, peptides, proteins, nucleic acids, membrane fractions, and polysaccharides.

The terms “pharmaceutically acceptable” or “pharmacologically acceptable,” as used herein, refer to compositions that do not substantially produce adverse reactions (e.g., toxic, allergic or immunological reactions) when administered to a subject.

As used herein, the term “pharmaceutically acceptable carrier” refers to any of the standard pharmaceutical carriers including, but not limited to, phosphate buffered saline solution, water, and various types of wetting agents (e.g., sodium lauryl sulfate), any and all solvents, dispersion media, coatings, sodium lauryl sulfate, isotonic and absorption delaying agents, disintrigrants (e.g., potato starch or sodium starch glycolate), polyethyl glycol, other natural and non-naturally occurring carries, and the like. The compositions also can include stabilizers and preservatives. Examples of carriers, stabilizers and adjuvants have been described and are known in the art (See e.g., Martin, Remington's Pharmaceutical Sciences, 15th Ed., Mack Publ. Co., Easton, Pa. (1975), incorporated herein by reference).

As used herein, the term “pharmaceutically acceptable salt” refers to any salt (e.g., obtained by reaction with an acid or a base) of a composition of the present disclosure that is physiologically tolerated in the target subject. “Salts” of the compositions of the present disclosure may be derived from inorganic or organic acids and bases. Examples of acids include, but are not limited to, hydrochloric, hydrobromic, sulfuric, nitric, perchloric, fumaric, maleic, phosphoric, glycolic, lactic, salicylic, succinic, toluene-p-sulfonic, tartaric, acetic, citric, methanesulfonic, ethanesulfonic, formic, benzoic, malonic, sulfonic, naphthalene-2-sulfonic, benzenesulfonic acid, and the like. Other acids, such as oxalic, while not in themselves pharmaceutically acceptable, may be employed in the preparation of salts useful as intermediates in obtaining the compositions of the present disclosure and their pharmaceutically acceptable acid addition salts. Examples of bases include, but are not limited to, alkali metal (e.g., sodium) hydroxides, alkaline earth metal (e.g., magnesium) hydroxides, ammonia, and compounds of formula NW₄⁺, wherein W is C₁₋₄ alkyl, and the like.

Examples of salts include, but are not limited to: acetate, adipate, alginate, aspartate, benzoate, benzenesulfonate, bisulfate, butyrate, citrate, camphorate, camphorsulfonate, cyclopentanepropionate, digluconate, dodecylsulfate, ethanesulfonate, fumarate, flucoheptanoate, glycerophosphate, hemisulfate, heptanoate, hexanoate, chloride, bromide, iodide, 2-hydroxyethanesulfonate, lactate, maleate, methanesulfonate, 2-naphthalenesulfonate, nicotinate, oxalate, palmoate, pectinate, persulfate, phenylpropionate, picrate, pivalate, propionate, succinate, tartrate, thiocyanate, tosylate, undecanoate, and the like. Other examples of salts include anions of the compounds of the present disclosure compounded with a suitable cation such as Na⁺, NH⁴⁺, and NW⁴⁺ (wherein W is a C₁₋₄ alkyl group), and the like. For therapeutic use, salts of the compounds of the present disclosure are contemplated as being pharmaceutically acceptable. However, salts of acids and bases that are non-pharmaceutically acceptable may also find use, for example, in the preparation or purification of a pharmaceutically acceptable compound.

As used herein, the terms “at risk for infection” and “at risk for disease” refer to a subject that is predisposed to experiencing a particular infection or disease (e.g., respiratory infection or disease). This predisposition may be genetic (e.g., a particular genetic tendency to experience the disease, such as heritable disorders), or due to other factors (e.g., immunosuppression, compromised immune system, immunodeficiency, environmental conditions, exposures to detrimental compounds present in the environment, etc.). Thus, it is not intended that embodiments of the present disclosure be limited to any particular risk (e.g., a subject may be “at risk for disease” simply by being exposed to and interacting with other people), nor is it intended that embodiments of the present disclosure be limited to any particular disease.

As used herein, the terms “% neutralization capacity” and “neutralizing antibody capacity” in general, and the terms “coronavirus % neutralization capacity” and “coronavirus neutralizing antibody capacity” more specifically, refer to the capacity of a sample to neutralize a coronavirus as determined using the competitive ELISA assay described in Example 2. The % neutralization capacity can be expressed in a variety of ways, including using a Sample Neutralization Index, as described herein. As described further herein, the % neutralization capacity of a sample can be used to detect or determine the amount of coronavirus neutralizing antibodies in a sample or the degree of neutralization of a sample, expressed using the % neutralization capacity metric (e.g., SNI), which is distinguishable from determining the coronavirus binding capacity of a sample (e.g., SCR).

As used herein, the terms “neutralizing antibody titer” and “coronavirus neutralizing antibody titer” refer to the amount of coronavirus neutralizing antibodies in a sample. As described further herein, the coronavirus neutralizing antibody titer of a sample can be determined from the % neutralization capacity of the sample (e.g., based on a positive control). Typically, determining the neutralization capacity of a sample (e.g., antibody or plasma/serum sample) involves the use of a plaque reduction neutralization test (PRNT), or a focus reduction neutralization test (FRNT), which quantify the neutralizing antibody titer for a virus (e.g., a coronavirus). The PRNT and the FRNT methods can both be used to determine the concentration of a sample (e.g., serum or antibody solution) required to reduce the number of plaques/foci by 50% compared to the sample-free virus, which provides a measure of how much antibody is present (antibody titer) or how effective it is. This measurement is denoted as the PRNT₅₀ or the FRNT₅₀ value.

### Coronavirus Neutralizing Antibodies

The present disclosure provides methods for detecting and quantifying coronavirus neutralizing antibody capacity and/or titer in a sample. Coronaviruses are named for the crown-like spikes on their surface. There are four main sub-groupings of coronaviruses, known as alpha, beta, gamma, and delta. Human coronaviruses were first identified in the mid-1960s. Seven coronaviruses have been identified that can infect people, they are: 229E (alpha coronavirus) NL63 (alpha coronavirus); OC43 (beta coronavirus); HKU1 (beta coronavirus); MERS-CoV (the beta coronavirus that causes Middle East Respiratory Syndrome, or MERS); SARS-CoV (the beta coronavirus that causes severe acute respiratory syndrome, or SARS); and SARS-CoV-2 (the novel coronavirus that causes coronavirus disease 2019, or COVID-19). Coronaviruses are a large family of viruses that are common in people and many different species of animals, including camels, cattle, cats, and bats. Rarely, animal coronaviruses can infect people and then spread between people such as with MERS-CoV, SARS-CoV, and SARS-CoV-2 (COVID-19). The SARS-CoV-2 virus is a betacoronavirus, like MERS-CoV and SARS-CoV. MERS-CoV and SARS-CoV have been known to cause severe illness in people. The complete clinical picture with regard to COVID-19 is not fully understood. Reported illnesses have ranged from mild to severe, including illness resulting in death. While information so far suggests that most COVID-19 illness is mild, a report out of China suggests serious illness occurs in 16% of cases. Older people and people with certain underlying health conditions like heart disease, lung disease and diabetes, for example, seem to be at greater risk of serious illness.

Neutralizing antibodies identified using the disclosed methods can specifically bind to any known or as yet undiscovered coronavirus, such as, for example, coronavirus OC43, coronavirus 229E, coronavirus NL63, coronavirus HKU1, MERS-CoV, SARS-CoV, or SARS-CoV-2 (COVID-19). In some embodiments, the neutralizing antibodies are directed against SARS-CoV-2 (COVID-19). In the context of the present disclosure a “neutralizing antibody” is an antibody that binds to a virus (e.g., a coronavirus) and interferes with the virus' ability to infect a host cell. Coronavirus spike proteins are known to elicit potent neutralizing-antibody and T-cell responses. The ability of a virus (e.g., coronavirus OC43, coronavirus 229E, coronavirus NL63, coronavirus HKU1, MERS-CoV, SARS-CoV, or SARS-CoV-2 (COVID-19)) to gain entry into cells and establish infection is mediated by the interactions of its Spike glycoproteins with human cell surface receptors. In the case of coronaviruses, Spike proteins are large type I transmembrane protein trimers that protrude from the surface of coronavirus virions. Each Spike protein comprises a large ectodomain (comprising S1 and S2), a transmembrane anchor, and a short intracellular tail. The 51 subunit of the ectodomain mediates binding of the virion to host cell-surface receptors through its receptor-binding domain (RBD). The S2 subunit fuses with both host and viral membranes, by undergoing structural changes.

SARS-CoV-2 utilizes the Spike glycoprotein to interact with cellular receptor ACE2 (Zhou et al., Nature 579: 270-273, doi:10.1038/s41586-020-2012-7 (2020); Hoffmann et al., Cell, 50092-8674(0020)30229-30224, doi:10.1016/j.cell.2020.02.052 (2020) doi:10.1016/j.cell.2020.02.052 (2020). The amino acid sequence of the SARS-CoV-2 spike protein has been deposited with the National Center for Biotechnology Information (NCBI) under Accession No. QHD43416. Binding with ACE2 triggers a cascade of cell membrane fusion events for viral entry. The high-resolution structure of SARSCoV-2 RBD bound to the N-terminal peptidase domain of ACE2 has recently been determined, and the overall ACE2-binding mechanism is virtually the same between SARS-CoV-2 and SARS-CoV RBDs, indicating convergent ACE2-binding evolution between these two viruses (Gui et al., CellRes 27, 119-129, doi:10.1038/cr.2016.152 (2017); Song et al., PLoS Pathog 14, e1007236-e1007236, doi:10.1371/journal.ppat.1007236 (2018); Yuan et al., Nat Commun 8, 15092-15092, doi:10.1038/ncomms15092 (2017); and Wan et al., J Virol, JVI.00127-00120, doi:10.1128/JVI.00127-20 (2020)). This indicates that disruption of the RBD and ACE2 interaction, e.g., by neutralizing antibodies, acts to block SARS-CoV-2 entry into the target cell. Indeed, a few such disruptive agents targeted to ACE2 have been shown to inhibit SARS-CoV infection (Kruse, R. L., F1000Res, 9: 72-72; doi:10.12688/f1000research.22211.2 (2020); and Li et al., Nature 426, 450-454; doi:10.1038/nature02145 (2003)). In addition, neutralizing antibodies directed against coronaviruses (also referred to herein as “coronavirus neutralizing antibodies”) have been identified and isolated (see, e.g., Liu et al., Potent neutralizing antibodies directed to multiple epitopes on SARS-CoV-2 spike. Nature (2020). doi.org/10.1038/s41586-020-2571-7; Rogers et al., Science 15 Jun. 2020:eabc7520; DOI: 10.1126/science.abc7520; Alsoussi et al., J Immunol Jun. 26, 2020, ji2000583; DOI: /doi.org/10.4049/jimmunol.2000583; Kreer et al., Cell, S0092-8674(20)30821-7. 13 Jul. 2020, doi:10.1016/j.cell.2020.06.044; Tai et al., J Virol. 2017 Jan. 1; 91(1): e01651-16; and Niu et al., J Infect Dis. 2018 Oct. 15; 218(8): 1249-1260).

### Samples and Subjects

The disclosed methods may be performed on any suitable sample. In some embodiments, the sample is obtained directly from a subject (e.g., a human), and comprises blood, plasma or immunoglobulin prepared therefrom, or serum. In other embodiments, the sample may be a fluid or solution obtained from a cell culture. In some embodiments, the sample comprises a pooled plasma composition comprising plasma samples from a plurality of human plasma donors. In one embodiment, the pooled plasma comprises plasma samples obtained from 50-3000 or more (e.g., more than 50, 100, 200, 300, 400, 500, 750, 1000, 1250, 1500, 1750, 2000, 2500, 3000, 3500, 4000 or more) human subjects. In another embodiment, the pooled plasma comprises plasma samples obtained from 100-1000 human subjects. In another embodiment, the pooled plasma comprises plasma samples obtained from at least 1000 human subjects. In one embodiment, the composition comprising pooled plasma samples further comprises a pharmaceutically acceptable carrier (e.g., natural and/or non-naturally occurring carriers). In one embodiment, the pooled plasma composition is utilized to prepare immune globulin (e.g., for intravenous administration to a subject).

Any suitable method for obtaining plasma, antibody samples, pooled plasma compositions and/or immunoglobulin from same are within the scope of the present disclosure. Further, any suitable method for producing, manufacturing, purifying, fractionating, enriching, etc., antibody samples and/or plasma pools is within the bounds of the present disclosure. Exemplary techniques and procedures for collecting antibody samples and producing plasma pools are provided, for example, in: U.S. Pat. Nos. 4,174,388; 4,346,073; 4,482,483; 4,587,121; 4,617,379; 4,659,563; 4,665,159; 4,717,564; 4,717,766; 4,801,450; 4,863,730; 5,505,945; 5,582,827; 6,692,739; 6,962,700; 6,984,492; 7,045,131; 7,488,486; 7,597,891; 6,372,216; U.S. Patent App. No. 2003/0118591; U.S. Patent App. No. 2003/0133929 U.S. Patent App. No. 2005/0053605; U.S. Patent App. No. 2005/0287146; U.S. Patent App. No. 2006/0110407; U.S. Patent App. No. 2006/0198848; U.S. Patent App. No. 2006/0222651; U.S. Patent App. No. 2007/0037170; U.S. Patent App. No. 2007/0249550; U.S. Patent App. No. 2009/0232798; U.S. Patent App. No. 2009/0269359; U.S. Patent App. No. 2010/0040601; U.S. Patent App. No. 2011/0059085; and U.S. Patent App. No. 2012/0121578; herein incorporated by reference in their entireties. Embodiments of the present disclosure may utilize any suitable combination of techniques, methods, or compositions from the above listed references.

In some embodiments, plasma samples are obtained from donor subjects in the form of donated or purchased biological material (e.g., blood or plasma). In some embodiments, blood or plasma samples are obtained from a commercial source. In some embodiments, a plasma sample, blood donation, or plasma donation is screened for pathogens, and either cleaned or discarded if particular pathogens are present. In one embodiment, screening occurs prior to pooling a donor sample with other donor samples. In other embodiments, screening occurs after pooling of samples. Antibodies, blood, and/or plasma may be obtained from any suitable subjects. In some embodiments, immunoglobulin, antibodies, blood, and/or plasma are obtained from a subject who has recently (e.g., within 1 year, within 6 months, within 2 months, within 1 month, within 2 weeks, within 1 week, within 3 days, within 2 days, within 1 day) been vaccinated against or been exposed to one or more specific pathogens. Pathogens to which a donor may have elevated titer of antibodies include, but are not limited to, respiratory syncytial virus (RSV) and coronavirus (e.g., coronavirus OC43, coronavirus 229E, coronavirus NL63, coronavirus HKU1, MERS-CoV, SARS-CoV, or SARS-CoV-2 (COVID-19)), or other human viral or bacterial pathogens.

In some embodiments, the present disclosure provides a composition comprising pooled plasma samples (e.g., a therapeutic composition) comprising plasma from a plurality of donors (e.g., 100 or more human donors), that have been clinically diagnosed with an infection by a viral or bacterial pathogen, such as an infection from one or more of a coronavirus (e.g., coronavirus OC43, coronavirus 229E, coronavirus NL63, coronavirus HKU1, MERS-CoV, SARS-CoV, SARS-CoV-2 (COVID-19)), respiratory syncytial virus (RSV), influenza A virus, influenza B virus, parainfluenza virus type 1, parainfluenza virus type 2, metapneumovirus, S. pneumonia, H. influenza, L. pneumophila, and group A Streptococcus. In some embodiments, a plurality of donors have recovered or are recovering from the viral infection. In some embodiments, a clinical diagnosis of a viral infection is carried out by a medical or laboratory professional and involves obtaining a sample(s) from the plurality of donors (e.g., blood sample, plasma sample, serum sample, fecal sample, urine sample cheek swab, sputum sample, and the like), and testing the sample using any of a variety of antibody-based and/or molecular (e.g., PCR) and/or clinical chemistry testing protocols to identify the presence of the virus and/or one or more physiological responses from the subject that correlates to the presence/absence of the virus. A clinical diagnosis generally involves a physiological readout based on the sample that indicates whether a subject has recovered or is recovering from the infection. A physiological indication of recovery can include, but is not limited to, presence/absence of an antibody, a nucleic acid, a metabolite, and the like. In some embodiments, a clinical diagnosis can indicate whether a subject has a coronavirus infection (e.g., coronavirus OC43, coronavirus 229E, coronavirus NL63, coronavirus HKU1, MERS-CoV, SARS-CoV, SARS-CoV-2 (COVID-19)), as well as whether the subject has recovered or is recovering from the coronavirus infection. In some embodiments, the one or more of the plurality of human plasma donors have been clinically diagnosed with infection by the coronavirus and have recovered from the infection. In some embodiments, the one or more of the plurality of human plasma donors have been clinically diagnosed with an infection from at least a second pathogen (e.g., virus or bacteria) and have recovered from the infection. The second pathogen may be, for example, respiratory syncytial virus (RSV), influenza A virus, influenza B virus, parainfluenza virus type 1, parainfluenza virus type 2, metapneumovirus, coronavirus OC43, coronavirus 229E, coronavirus NL63, coronavirus HKU1, MERS-CoV, SARS-CoV, SARS-CoV-2 (COVID-19), S. pneumonia, H. influenza, L. pneumophila, and group A Streptococcus. In some embodiments, the one or more of the plurality of human plasma donors have not been clinically diagnosed with infection by a coronavirus. In some embodiments, the one or more of the plurality of human plasma donors have not been clinically diagnosed with an infection from the at least second pathogen. In some embodiments, the one or more of the plurality of human donors have been selected based on at least one pre-preselection criterion, including but not limited to occupation (e.g., teacher, flight attendant, healthcare professional), proximity to an infection hotspot, degree of contact to other humans, and the like.

In some embodiments, the subject is at elevated risk for infection (e.g., by one or multiple specific pathogens (e.g., respiratory pathogens)). The subject may be a neonate. In some embodiments, the subject has an immunodeficiency (e.g., a subject receiving immunosuppressing drugs (e.g., a transplant patient), suffering from a disease of the immune system, suffering from a disease that depresses immune functions, undergoing a therapy (e.g., chemotherapy) that results in a suppressed immune system, experiencing an extended hospital stay, and/or a subject anticipating direct exposure to a pathogen or pathogens. In some embodiments, the subject may have a healthy or normal immune system. In some embodiments, the subject is one that has a greater than normal risk of being exposed to a pathogen (e.g., a coronavirus). In some embodiments, the subject is a soldier, an emergency responder or other subject that has a higher than normal risk of being exposed to a pathogen (e.g., a coronavirus).

### Neutralization Assay Methods

The present disclosure provides methods of detecting coronavirus neutralizing antibodies in a sample which, in some embodiments, comprise first contacting a sample with a solid support comprising a coronavirus cell receptor immobilized thereto to form a mixture, and subsequently contacting the mixture with a conjugate comprising a reporter molecule attached to a peptide comprising a receptor binding domain (RBD) of a coronavirus spike protein, whereby, if coronavirus neutralizing antibodies are present in the sample, the RBD binds to the coronavirus neutralizing antibodies and does not bind to the immobilized coronavirus cell receptor. In other embodiments, the methods comprise first contacting a sample with a conjugate comprising a reporter molecule attached to a peptide comprising a receptor binding domain (RBD) of a coronavirus spike protein to form a mixture, wherein the RBD binds to coronavirus neutralizing antibodies if present in the sample, and subsequently contacting the mixture with a solid support comprising a coronavirus cell receptor immobilized thereto, whereby, if coronavirus neutralizing antibodies are present in the sample, the RBD bound to the coronavirus neutralizing antibodies does not bind to the immobilized coronavirus cell receptor.

The terms “solid phase” and “solid support” are used interchangeably herein and refer to any material that can be used to attach and/or attract and immobilize one or more proteins or other specific binding members. The term “immobilized,” as used herein, refers to a stable association of a binding member with a surface of a solid support. Any solid support known in the art can be used in the kits and methods described herein, including but not limited to, solid supports made out of polymeric materials in the form of planar substrates or beads. Examples of suitable solid supports include electrodes, test tubes, beads, a polystyrene micro-titer plate, a multiplexing chip array, polystyrene bead particles, magnetic particles, a cellulose membrane, microparticles, nanoparticles, wells of micro- or multi-well plates, gels, colloids, biological cells, sheets, and chips. The terms “bead” and “particle” are used herein interchangeably and refer to a substantially spherical solid support. A microparticle may be between about 0.1 nm and about 10 microns (e.g., between about 50 nm and about 5 microns, between about 100 nm and about 1 micron, between about 0.1 nm and about 700 nm, between about 500 nm and about 10 microns, between about 500 nm and about 5 microns, between about 500 nm and about 3 microns, between about 100 nm and 700 nm, or between about 500 nm and 700 nm). Nanoparticles are particles less than about 500 nm.

In certain embodiments, the solid support may be a magnetic bead or a magnetic particle. Magnetic beads/particles may be ferromagnetic, ferrimagnetic, paramagnetic, superparamagnetic or ferrofluidic. Exemplary ferromagnetic materials include Fe, Co, Ni, Gd, Dy, CrO₂, MnAs, MnBi, EuO, NiO/Fe. Examples of ferrimagnetic materials include NiFe₂O₄, CoFe₂O₄, Fe₃O₄ (or FeO.Fe₂O₃). Beads can have a solid core portion that is magnetic and is surrounded by one or more non-magnetic layers. Alternatively, the magnetic portion can be a layer around a non-magnetic core. The solid support on which a binding member is immobilized may be stored in dry or liquid form. The magnetic beads may be subjected to a magnetic field prior to or after contacting with the sample with a magnetic bead on which a binding member is immobilized.

The solid support may be contacted with a sample (e.g., a plasma sample) using any suitable method known in the art. The term “contacting,” as used herein, refers to any type of combining action which brings a specific binding member immobilized thereon into sufficiently close proximity with a sample such that a binding interaction will occur if a molecule or compound specific for the binding member is present in the sample. Contacting may be achieved in a variety of different ways, including combining the sample with microparticles or exposing target molecules to microparticles comprising binding members by introducing the microparticles in close proximity to the target molecules. The contacting may be repeated as many times as necessary.

In some embodiments, a coronavirus cell receptor may be attached to a solid support via a linkage, which may comprise any moiety, functionalization, or modification of the support and/or receptor protein that facilitates the attachment of the receptor to the solid support. The linkage between the receptor and the solid support may include one or more chemical or physical (e.g., non-specific attachment via van der Waals forces, hydrogen bonding, electrostatic interactions, hydrophobic/hydrophilic interactions; etc.) bonds and/or chemical spacers providing such bond(s). Any number of techniques may be used to attach a polypeptide (e.g., a receptor) to a wide variety of solid supports (see, e.g., U.S. Pat. Nos. 5,620,850; 5,624,711; Heller, Acc. Chem. Res., 23: 128 (1990); and Legua et al., Chromatographia, 58: 15-27(2003)).

In some embodiments, the coronavirus cell receptor is generated or engineered using routine molecular biology techniques, such as those described in, e.g., Sambrook, J., Molecular Cloning: A Laboratory Manual, Cold Spring Harbor Laboratory Press; 4th edition (Jun. 15, 2012); and Ausubel et al., eds., Short Protocols in Molecular Biology, 5th ed., John Wiley & Sons, Inc., Hoboken, N.J. (2002)). In this regard, the coronavirus cell receptor or portion thereof employed in the disclosed methods may be based upon or derived from any cell receptor utilized by any coronavirus. As discussed above, both the SARS-CoV and SARS-CoV-2 viruses utilize the ACE2 receptor to enter infected cells. MERS-CoV utilizes the dipeptidyl peptidase 4 (DPP4) receptor for cell entry. Other coronavirus cell receptors include, but are not limited to, aminopeptidase N (APN), carcinoembryonic antigen-related cell adhesion molecule 1 (CEACAM1), and sugar (Li, F., Journal of Virology, 89(4): 1954-1964 (2015); DOI: 10.1128/JVI.02615-14). In some embodiments, the coronavirus cell receptor is a recombinant ACE2 receptor (e.g., an ACE2 protein having the amino acid sequence deposited with the NCBI under Accession No. Q9BYF1), or a portion or fragment thereof. The disclosure is not limited to any particular recombinant coronavirus receptor. In some embodiments, recombinant ACE2 used in the assays of the present disclosure binds to a receptor binding domain of a coronavirus spike protein (e.g., a spike protein comprising an amino acid sequence deposited with the NCBI under Accession No. QHD43416) or a portion or fragment thereof.

When the method comprises applying the sample to a solid support having a coronavirus cell receptor immobilized thereto to form a mixture, the method subsequently comprises contacting the mixture with a conjugate comprising a reporter molecule attached to a protein or peptide comprising a receptor binding domain (RBD) of a coronavirus spike protein. The term “conjugate” refers to a binding protein (e.g., a peptide or antibody) chemically linked to a second chemical moiety. The term “reporter molecule,” as used herein, refers to a moiety that can produce a signal that is detectable by visual or instrumental means. Assays of the present disclosure are not limited by the means of detection or visualization. For example, the reporter molecule may be, for example, a signal-producing substance, such as a detectable tag (e.g., a fluorescent tag or label) a chromagen, an enzyme, a chemiluminescent compound, a radioactive compound, gold particles, organic dyes, etc. Enzyme reporters include, for example, horseradish peroxidase, luciferase, and alkaline phosphatase. Examples of suitable fluorescent compounds include, but are not limited to, 5-fluorescein, 6-carboxyfluorescein, 3′6-carboxyfluorescein, 5(6)-carboxyfluorescein, 6-hexachloro-fluorescein, 6-tetrachlorofluorescein, fluorescein isothiocyanate, rhodamine, phycobiliproteins, phycoerythrin, R-phycoerythrin, and allophycocyanin), quantum dots (e.g., zinc sulfide-capped cadmium selenide), a thermometric label, or an immuno-polymerase chain reaction label. In some embodiments, acridinium compounds may be used for chemiluminescence detection. Detectable labels, labeling procedures, and detection of labels are described in Polak and Van Noorden, Introduction to Immunocytochemistry, 2nd ed., Springer Verlag, N.Y. (1997), and in Haugland, Handbook of Fluorescent Probes and Research Chemicals (1996), Molecular Probes, Inc., Eugene, Oreg.

The reporter molecule can be conjugated to the coronavirus attachment moiety (e.g., RBD peptide) either directly or through a coupling or crosslinking agent. An example of a coupling agent that can be used is EDAC (1-ethyl-3-(3-dimethylaminopropyl) carbodiimide, hydrochloride), which is commercially available from Sigma-Aldrich, St. Louis, Mo. Other coupling agents that can be used are known in the art, and typically include disulfide groups, thioether groups, acid labile groups, photolabile groups, peptidase labile groups, and esterase labile groups. Crosslinking agents also include charged linkers, cleavable linkers, non-cleavable linkers, hydrophilic linkers, and dicarboxylic acid-based linkers. Additionally, many reporter molecules can be purchased or synthesized that already contain end groups that facilitate the coupling of the reporter molecule to the peptide.

The coronavirus attachment moiety or peptide (e.g., peptide comprising a receptor binding domain (RBD) of a coronavirus spike protein) may be prepared using routine molecular biology techniques, such as those disclosed herein. The nucleic acid and amino acid sequences of RBDs of various coronavirus spike proteins are known in the art (see, e.g., Tai et al., Cell Mol Immunol 17, 613-620 (2020). doi.org/10.1038/s41423-020-0400-4; and Chakraborti et al., Virology Journal volume 2, Article number: 73 (2005); and Chen et al., Biochemical and Biophysical Research Communications, 525(1): 135-140 (2020)). An exemplary RBD domain of a SARS-CoV-2 spike protein comprises the following amino acid sequence:

(SEQ ID NO: 1)
RVQPTESIVRFPNITNLCPFGEVFNATRFASVYAWNRKRISNCVADYSV
LYNSASFSTFKCYGVSPTKLNDLCFTNVYADSFVIRGDEVRQIAPGQTG
KIADYNYKLPDDFTGCVIAWNSNNLDSKVGGNYNYLYRLFRKSNLKPFE
RDISTEIYQAGSTPCNGVEGFNCYFPLQSYGFQPTNGVGYQPYRVVVLS
FELLHAPATVCGPKKSTNLVKNKCVNFNFNGLTGTGVLTESNKKFLPFQ
QFGRDIADTTDAVRDPQTLEILDITPCS.

As discussed above, in other embodiments, the methods described herein may comprise first contacting the sample with the conjugate comprising a reporter molecule attached to a peptide comprising a receptor binding domain (RBD) of a coronavirus spike protein to form a mixture, and then subsequently contacting the mixture with a solid support comprising a coronavirus cell receptor immobilized thereto. Whatever format is chosen (e.g., sample contacting the solid support followed by the conjugate or sample contacting the conjugate followed by the solid support), the sample is incubated with the solid support or the conjugate under conditions whereby coronavirus neutralizing antibodies, if present in the sample, compete with the coronavirus cell receptor for binding to the RBD on the conjugate, thereby preventing the RBD from binding to the coronavirus cell receptor immobilized on the solid support.

In one embodiment, contact between the sample, the solid support, and conjugate is maintained (i.e., incubated) for a sufficient period of time to allow for the binding interaction between coronavirus neutralizing antibodies (if present in the sample) and the RBD, and/or the RBD to the immobilized coronavirus cell receptor to occur. In this regard, for example, the sample may be incubated on a solid support or with the conjugate for at least 30 seconds and may last for 10 minutes or more. For example, the sample may be incubated with the solid support or the conjugate for about 1, 2, 3, 4, 5, 6, 7, 8, 9, 10 or more minutes. In addition, the incubating may be in a binding buffer that facilitates the specific binding interaction, such as, for example, albumin (e.g., BSA), non-ionic detergents (Tween-20, Triton X-100), and/or protease inhibitors (e.g., PMSF). The binding affinity and/or specificity of coronavirus neutralizing antibodies present in the sample may be manipulated or altered in the assay by varying the binding buffer. In some embodiments, the binding affinity and/or specificity may be increased or decreased by varying the binding buffer. Other conditions for the binding interaction, such as, for example, temperature and salt concentration, may also be determined empirically or may be based on manufacturer's instructions. For example, the contacting may be carried out at room temperature (21° C.-28° C., e.g., 23° C.-25° C.), 37° C., or 4° C.

Following an incubation under conditions that allow for sufficient binding of coronavirus neutralizing antibodies (if present in the sample) to the RBD, and/or the RBD to the immobilized coronavirus cell receptor to occur, any unbound sample and/or conjugate is washed away and complexes comprising the coronavirus cell receptor bound to the conjugate (via the RBD) remain immobilized on the solid support. Subsequently, the disclosed methods involve detecting and quantifying a signal from the reporter molecule using techniques known in the art. For example, if an enzymatic label is used, the labeled complex is reacted with a substrate for the enzymatic label that gives a quantifiable reaction, such as the development of color (e.g., 3,3′,5,5;-tetramethylbenzidine (TMB) for horseradish peroxidase). If the label is a radioactive label, the label is quantified using appropriate means, such as a scintillation counter. If the reporter molecule is a fluorescent tag or label, the label is quantified by stimulating the label with a light of one color (which is known as the “excitation wavelength”) and detecting another color (which is known as the “emission wavelength”) that is emitted by the label in response to the stimulation. If the reporter molecule is a chemiluminescent label, the label is quantified by detecting the light emitted either visually or by using luminometers, x-ray film, high speed photographic film, a CCD camera, etc. Once the amount of the reporter molecule has been quantified, the concentration of RBD-binding coronavirus neutralizing antibodies in the sample is determined by appropriate means, such as by use of a standard curve that has been generated using serial dilutions of neutralizing antibodies of known concentration. In other embodiments, a standard curve can be generated gravimetrically, by mass spectroscopy, or by other techniques known in the art.

In some embodiments, the intensity of the signal generated is directly proportional to the degree to which the RBD of the conjugate binds to the coronavirus cell receptor immobilized on the solid support. At the same time, the intensity of the signal generated is inversely proportional to the amount of neutralizing anti-RBD antibodies present in the sample. In other words, in the absence of coronavirus neutralizing antibodies in the sample, the ACE2 receptor immobilized on the solid support is available for binding by the RBD-containing conjugate, allowing detection of a signal from the reporter molecule. In the presence of coronavirus neutralizing antibodies, however, the neutralizing antibodies will bind to the RBD on the conjugate, and RBD binding to the ACE2 receptor is reduced or completely inhibited. In such cases, reduced signal—or no signal—will be detected from the solid support.

In some embodiments, the methods described herein are performed using any suitable assay. Suitable assays which may be employed include flow cytometry assays, competitive assays, inhibition assays, immunofluorescence assays, enzyme-linked immunosorbent (ELISA) assays, lateral flow assays, sandwich assays, and neutralization assays. In some embodiments, the methods described herein are performed using an enzyme linked immunosorbent assay (ELISA). ELISA (also referred to in the art as an “enzyme immunoassay” (EIA)) is a plate-based assay technique designed for detecting and quantifying soluble substances such as peptides, proteins, antibodies, and hormones in a sample. In an ELISA, a target macromolecule (e.g., a cell receptor) is immobilized on a solid surface (e.g., a microplate) and then complexed with a binding member specific for the target (e.g., a ligand) that is linked to a reporter enzyme. Detection is accomplished by measuring the activity of the reporter enzyme via incubation with the appropriate substrate to produce a measurable product (e.g., absorbance, chemiluminescence, fluorescence, or other visual signal). In the context of the present disclosure, an ELISA may be performed in either competitive or non-competitive formats. In some embodiments, the ELISA is a competitive inhibition assay (also referred to in the art as “inhibition ELISA” or “competitive immunoassay”) which enables the screening of inhibitory proteins by measuring the concentration of a potential inhibitor protein (e.g., a neutralizing antibody) by detection of signal interference. In the context of the present disclosure, coronavirus neutralizing antibodies present in the sample compete with the coronavirus cell receptor, which is pre-coated on a solid support (e.g., a multi-well plate), for binding to the RBD-containing conjugate. Depending on the amount of neutralizing antibodies in the sample, more or less free conjugate will be available to bind the coronavirus cell receptor (e.g., ACE2). Therefore, as discussed above, the more neutralizing antibodies present in the sample, the less binding of the conjugate to the cell receptor will be detected and the weaker the signal. Systems and methods for performing ELISA are known in the art and commercially available (see, e.g., Methods in Immunodiagnosis, 2nd Edition, Rose and Bigazzi, eds., John Wiley and Sons, 1980 and Campbell et al., Methods of Immunology, W. A. Benjamin, Inc., 1964). The assays described herein desirably are performed in the absence of cells or viruses (i.e., “cell-free” or “virus-free” assays).

In some embodiments, the disclosed methods desirably include positive and/or negative controls. A control may be analyzed concurrently with the sample from the subject, or a control may be analyzed before or after the sample has been analyzed using the disclosed methods. The results obtained from the sample can be compared to the results obtained from the control(s). Standard curves for the controls may be provided, with which assay results for the sample may be compared. Controls may be used to determine coronavirus neutralizing antibody capacity and/or titer. Thus, to confirm the presence of coronavirus neutralizing antibodies in the sample and/or to determine the amount of coronavirus neutralizing antibodies that are present in the sample, the disclosed methods may be performed with a positive control comprising a panel of one or more coronavirus neutralizing monoclonal antibodies, and the quantified signal of the positive control may be compared to the quantified signal of the sample. The panel of antibodies in the positive control may comprise one, two or more, three or more, four or more, or at least five coronavirus neutralizing antibodies. As discussed above, numerous neutralizing antibodies directed against several types of coronaviruses have been isolated, and any of these neutralizing antibodies may be included in the positive control panel. Exemplary coronavirus neutralizing antibodies that may be included in the positive control panel are disclosed in, for example, Zost et al., Rapid isolation and profiling of a diverse panel of human monoclonal antibodies targeting the SARS-CoV-2 spike protein. Nat Med (2020). doi.org/10.1038/s41591-020-0998-x. In other embodiments, the method further comprises comparing the results obtained from the sample with the results obtained using a negative control. The negative control may comprise at least one antibody that does not neutralize coronavirus infection (i.e., “coronavirus non-neutralizing antibodies”). The negative control may comprise a panel of two or more, three or more, four or more, or at least five coronavirus non-neutralizing antibodies.

The disclosure further provides a method of identifying plasma comprising coronavirus neutralizing antibodies. In some embodiments, the method comprises (a) contacting a plasma sample with a solid support comprising a coronavirus cell receptor immobilized thereto to form a mixture; (b) contacting the mixture with a conjugate comprising a reporter molecule attached to a peptide comprising a receptor binding domain (RBD) of a coronavirus spike protein, whereby, if coronavirus neutralizing antibodies are present in the plasma sample, the RBD binds to the coronavirus neutralizing antibodies and does not bind to the immobilized coronavirus cell receptor; (c) detecting and quantifying a signal from the reporter molecule, wherein the amount of detected signal is inversely proportional to the amount of coronavirus neutralizing antibodies present in the plasma sample; and (d) performing steps (a)-(c) on a positive control comprising a panel of one or more coronavirus neutralizing monoclonal antibodies instead of the plasma sample, and comparing the quantified signal of the positive control to the quantified signal of the plasma sample to determine the amount of coronavirus neutralizing antibodies that are present in the plasma sample (e.g., to detect the presence of coronavirus neutralizing antibodies and/or to quantify the coronavirus neutralizing antibody titer). In other embodiments, the method of identifying plasma comprising coronavirus neutralizing antibodies comprises: (a) contacting a plasma sample with a conjugate comprising a reporter molecule attached to a peptide comprising a receptor binding domain (RBD) of a coronavirus spike protein to form a mixture, wherein the RBD binds to coronavirus neutralizing antibodies if present in the plasma sample; (b) contacting the mixture with a solid support comprising a coronavirus cell receptor immobilized thereto, whereby, if coronavirus neutralizing antibodies are present in the plasma sample, the RBD bound to the coronavirus neutralizing antibodies does not bind to the immobilized coronavirus cell receptor; (c) detecting and quantifying a signal from the reporter molecule, wherein the amount of detected signal is inversely proportional to the amount of coronavirus neutralizing antibodies present in the plasma sample; and (d) performing steps (a)-(c) on a positive control comprising a panel of one or more coronavirus neutralizing monoclonal antibodies instead of the plasma sample, and comparing the quantified signal of the positive control to the quantified signal of the plasma sample to determine the amount of coronavirus neutralizing antibodies that are present in the plasma sample (e.g., to detect the presence of coronavirus neutralizing antibodies and/or to quantify the coronavirus neutralizing antibody titer). Descriptions of the plasma sample, solid support, conjugate, controls, and components thereof set forth above in connection with the methods of detecting coronavirus neutralizing antibodies also are applicable to the methods of identifying plasma comprising coronavirus neutralizing antibodies. The disclosure also provides a method of identifying a subject (e.g., a subject that has been vaccinated with a vaccine specific for the coronavirus or a subject that has recovered from coronavirus infection) harboring coronavirus neutralizing antibodies and/or quantifying the titer of coronavirus neutralizing antibodies in a subject, the method comprising performing any of the above-described methods on a sample obtained from the subject (e.g., a sample comprising plasma).

The above-described methods may be utilized in a variety of applications, including to determine the efficacy of immunization or vaccination against a coronavirus (e.g., determining the levels of coronavirus-specific antibody titers). In such an embodiment, the disclosed methods are performed on a sample from a subject has been vaccinated with a vaccine specific for the coronavirus. The methods also may be employed to screen plasma for its ability to provide protection from coronavirus infection, as well as for therapeutic applications (e.g., convalescent plasma).

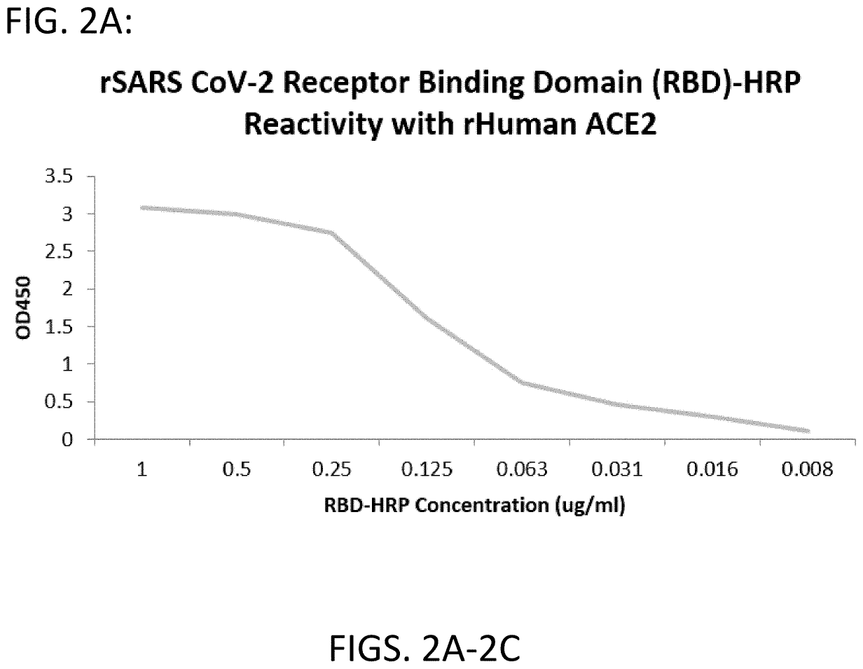

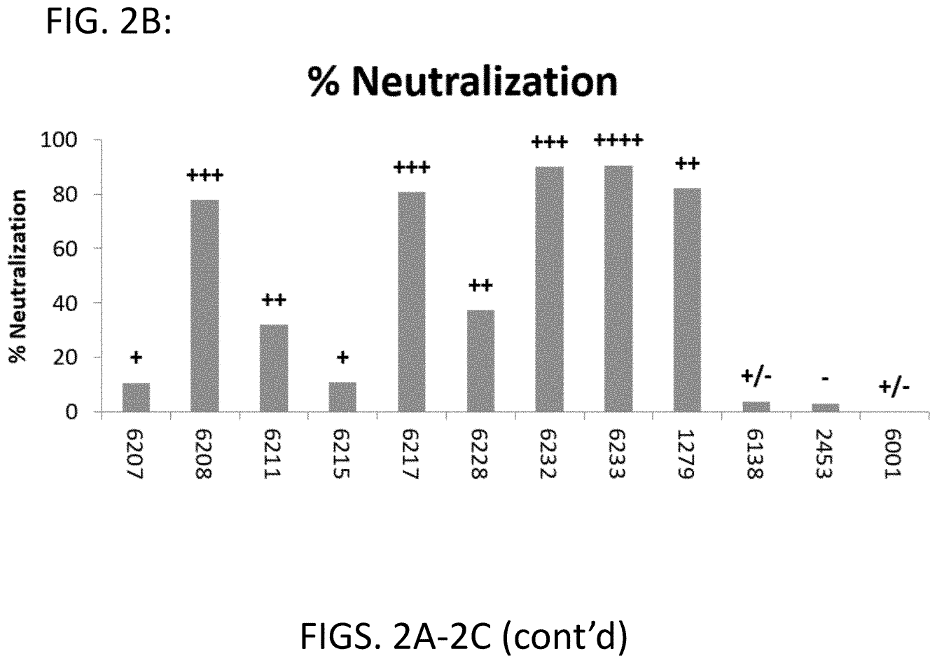

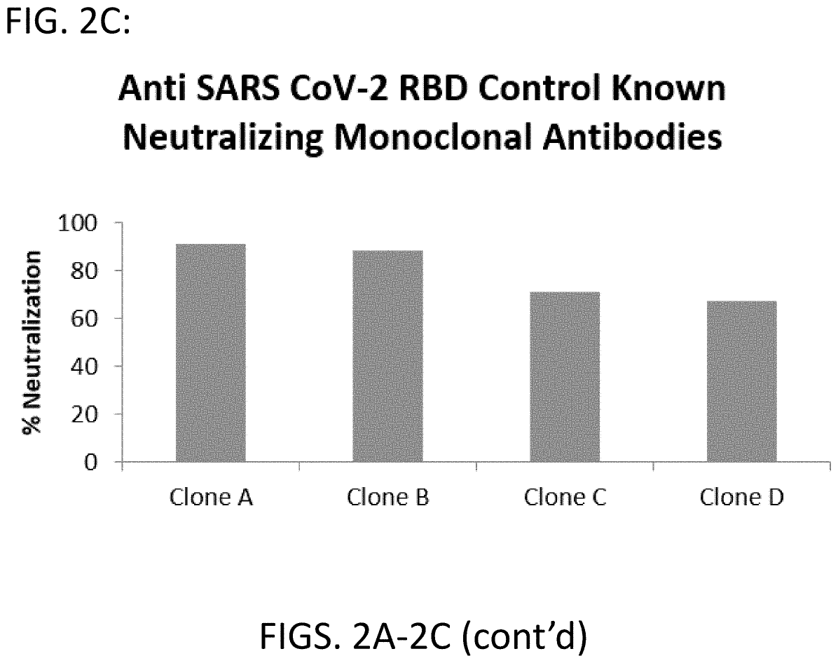

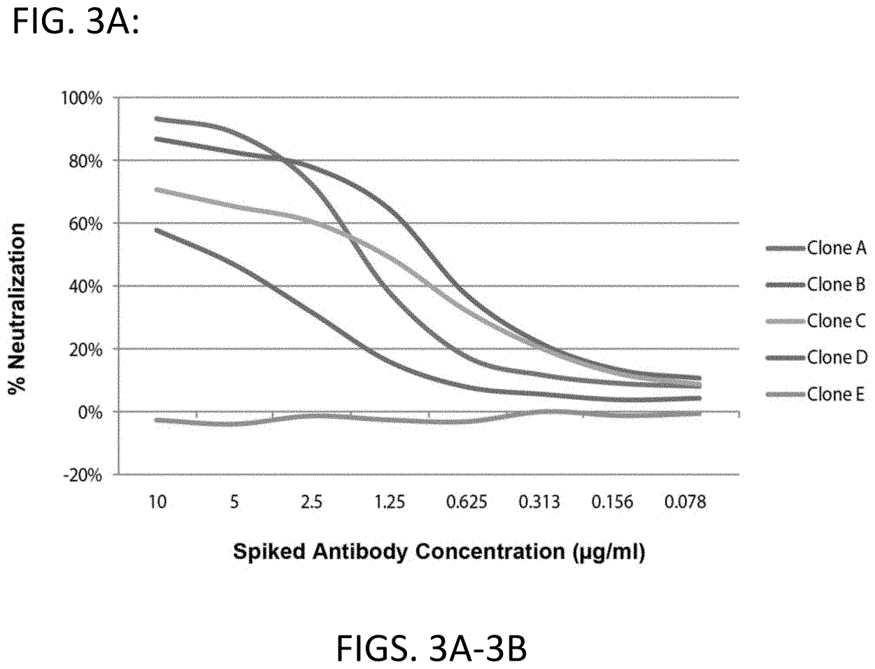

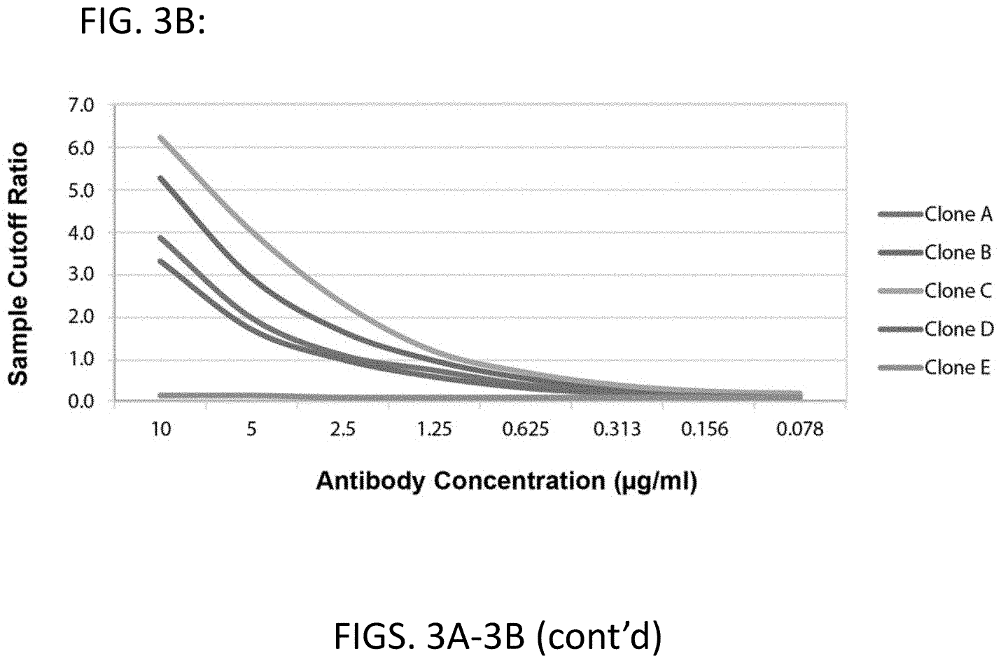

In accordance with the methods described herein, the terms “% neutralization capacity” and “% neutralization” refer to the capacity of a sample to neutralize a coronavirus as determined using the competitive ELISA assay described in Example 2. The % neutralization capacity can be expressed in a variety of ways, including using a Sample Neutralization Index, as described herein. The methods of the present disclosure provide an alternative means for detecting or determining the amount of neutralizing antibodies in a sample or the degree of neutralization of a sample, expressed using the % neutralization capacity metric, that is distinguishable from determining the binding capacity of a sample. As demonstrated in FIGS. 2A, 3A, and 3B, as well as Table 6, binding capacity (e.g., expressed as Sample Cutoff Ratio) and % neutralization capacity (e.g., expressed as SNI) of any given sample are independent and not necessarily correlative.

Additionally, the methods of the present disclosure provide a means for determining neutralization titers based on the % neutralization capacity of a sample. Traditionally, determining the neutralization capacity of a sample (e.g., antibody or plasma/serum sample) involves the use of a plaque reduction neutralization test (PRNT), which quantifies the titer of neutralizing antibody for a virus. For example, a serum sample or a solution of antibody to be tested is diluted and mixed with a viral suspension. This mixture is then incubated to allow the antibody to react with the virus, and subsequently poured over a confluent monolayer of host cells. The surface of the cell layer is covered in a layer of agar or carboxymethyl cellulose to prevent the virus from spreading indiscriminately. The concentration of plaque forming units can be estimated by the number of plaques (regions of infected cells) formed after a certain period of time. Depending on the virus, the plaque forming units can be measured by microscopic observation, fluorescent antibodies or specific dyes that react with infected cells. For example, the focus reduction neutralization test (FRNT) is a variation of the plaque assay, but instead of relying on cell lysis in order to detect plaque formation, an FRNT assay uses fluorescently labeled antibodies specific for a viral antigen to detect infected host cells and infectious virus particles before an actual plaque is formed. The FRNT is useful for quantifying classes of viruses that do not lyse the cell membranes, as these viruses would not be amenable to the plaque assay. The PRNT and the FRNT methods both determine the concentration of a sample (e.g., serum or antibody solution) required to reduce the number of plaques/foci by 50% compared to the sample-free virus, which provides a measure of how much antibody is present (antibody titer) or how effective it is. This measurement is denoted as the PRNT₅₀ or the FRNT₅₀ value.

As provided herein, the methods of the present disclosure for determining the % neutralization capacity of a sample can also be used to determine the neutralizing antibody titer present in the sample. For example, the % neutralization capacity of a sample can be expressed as SNI, which indicates whether the sample has a % neutralization capacity above a positive control, below a positive control, or substantially similar to a positive control (see, e.g., Example 2). By obtaining, for example, the FRNT50 neutralizing antibody titer of the positive control, it is possible to determine the corresponding FRNT50 neutralizing antibody titer of the sample based on their corresponding % neutralization capacities, as determined using the methods of the present disclosure. As described further herein, the methods of the present disclosure for determining % neutralization capacity of a sample do not require use of live viruses or cells, which is one advantage over that of PRNT and FRNT. That is, the methods of the present disclosure can be performed in a matter of hours and with much less resources because the assay is not dependent on the growth of cells, a particular cell type, or the high demands handling of live viruses.

Additionally, the ability to determine % neutralization capacity of a given sample based on a positive control provides a means for quickly and accurately assessing any sample that may contain coronavirus neutralizing antibodies. As described further herein, the sample can be an individual plasma or serum sample from a subject. In some embodiments, the sample is from a subject that has been infected with a coronavirus, exposed to a coronavirus, or is suspected of being infected or exposed to a coronavirus. Determining % neutralization capacity in such samples can determine whether sufficient neutralizing antibodies are present in the subject or whether certain treatment is needed. In some embodiments, the subject may have been administered a coronavirus vaccine or other therapeutic agent and the determining % neutralization capacity can be used to assess whether the subject has sufficient neutralizing antibodies or whether additional treatment is warranted. In some embodiments, determining the % neutralization capacity of a sample indicates that a subject has a high titer of coronavirus antibodies after being administered a coronavirus vaccine or being infected with a coronavirus. In such cases, the sample from the subject can be used as a positive control in accordance with the methods described above. In some embodiments, the positive control sample can be included as part of a kit for determining % neutralization capacity and neutralizing antibody titers. In some embodiments, the neutralizing antibody titer of the positive control has been determined (e.g., using PRNT or FRNT), and this information is included in the kit.

The methods of the present disclosure can be used to determine the % neutralization capacity of donor samples (either individually or as a pooled mixture) for use as a convalescent plasma composition that can be used to effectively treat a coronavirus infection. In some embodiments, the % neutralization capacity of one or more samples can be determined individually or as a pooled mixture. If the % neutralization capacity of the samples or pooled mixture of samples indicates a sufficiently high titer, these samples can be used to generate convalescent plasma to be administered as a therapeutic to treat a coronavirus infection. In contrast to assays that measure binding capacity, the methods of the present disclosure can be used to determine the specific levels of % neutralization capacity and neutralizing antibody titers, which can serve as the basis for generating a therapeutic convalescent plasma composition with a specific % neutralization capacity and neutralizing antibody titer.

The assays of the present disclosure can also be used to determine the % neutralization capacity of a sample in a semi-quantitative or quantitative manner. For example, as shown in Example 7, the % SNI value of a test sample (e.g., serum or plasma) can be compared to a range of different % SNI values that correspond to reference samples with different degrees of % neutralization capacity and/or antibody titers. Depending on the criteria used to characterize or group the reference samples (e.g., % neutralization capacity, antibody titer, vaccination status, and the like), the test sample can be determined to have low, medium, or high % neutralization capacity or antibody titer based on the reference samples. In some embodiments, the reference samples can be obtained from subjects that have a certain coronavirus vaccination status (e.g., vaccinated or unvaccinated) or have recovered from a coronavirus infection. These samples can be used as control samples and serve as the basis for the reference samples. This semi-quantitative approach to determining the coronavirus % neutralization capacity or antibody titer can be used as a basis by which a test sample (and corresponding test subject) can be characterized as a potential donor for generating a convalescent plasma therapeutic composition and/or to determine the neutralizing antibody capacity or antibody titer present in a sample (e.g., if a subject has a protective level of coronavirue neutralizing antibodies, for example, acquired via natural infection or via immunization).

In some embodiments, quantitative assessments of a test sample for coronavirus neutralizing antibody capacity or antibody titer can be performed by diluting the sample and carrying out the assay described in Example 2. As provided in Example 8, a series of sample dilutions can be made and the % neutralization capacity can be determined for each of the dilutions. A quantitative determination of its coronavirus neutralizing antibody capacity or antibody titer can be made by determining whether the % SNI is above that cutoff for each dilution. For example, a cutoff value can be set at 20% (e.g., a sample with an SNI of 20% and over was determined to be positive for coronavirus neutralizing antibodies), and the sample can be diluted to 1:10, 1:80, 1:320, and 1:1280. The % SNI can then be determined on each of these dilutions using the neutralization assays of the present disclosure. For example, a positive % SNI value for the 1:1280 dilution would indicate a high coronavirus titer in that sample. A positive % SNI value for the 1:80 or the 1:320 dilution, but not the 1:1280 dilution, would indicate a medium coronavirus titer in that sample. A positive % SNI value for the 1:10 dilution, but not the 1:80 dilution, the 1:320 dilution, or the 1:1280 dilution, would indicate a low coronavirus titer in that sample. This quantitative approach to determining the coronavirus % neutralization capacity or antibody titer can be used as a basis by which a test sample (and corresponding test subject) can be characterized as a potential donor for generating a convalescent plasma therapeutic composition and/or to determine the neutralizing antibody capacity or antibody titer present in a sample (e.g., if a subject has a protective level of coronavirue neutralizing antibodies, for example, acquired via natural infection or via immunization).

As one of ordinary skill in the art would readily understand based on the present disclosure, the number of dilutions and/or the range of dilutions can be adjusted based, for example, on the samples and the assay design. In some embodiments, samples can be diluted in any number, manner, or amount from 1:1 to 1:50,000 or more. In some embodiments, samples can be diluted in any number, manner, or amount from 1:10 to 1:10,000. In some embodiments, samples can be diluted in any number, manner, or amount from 1:100 to 1:1,000. In some embodiments, samples can be diluted in any number, manner, or amount from 1:10 to 1:2,000, 1:10 to 1:3,000, 1:10 to 1:4,000, 1:10 to 1:5,000, 1:10 to 1:6,000, 1:10 to 1:7,000, 1:10 to 1:8,000, or 1:10 to 1:9,000. In some embodiments, samples can be diluted in any number, manner, or amount from 1:100 to 1:2,000, 1:100 to 1:3,000, 1:100 to 1:4,000, 1:100 to 1:5,000, 1:100 to 1:6,000, 1:100 to 1:7,000, 1:100 to 1:8,000, 1:100 to 1:9,000, 1:100 to 1:10,000, 1:100 to 1:11,000, 1:100 to 1:12,000, 1:100 to 1:13,000, 1:100 to 1:14,000, 1:100 to 1:15,000, 1:100 to 1:16,000, 1:100 to 1:17,000, 1:100 to 1:18,000, 1:100 to 1:19,000, or 1:100 to 1:20,000. In some embodiments, samples can be diluted in any number, manner, or amount from 1:1000 to 1:2,000, 1:1000 to 1:3,000, 1:1000 to 1:4,000, 1:1000 to 1:5,000, 1:1000 to 1:6,000, 1:1000 to 1:7,000, 1:1000 to 1:8,000, 1:1000 to 1:9,000, 1:1000 to 1:10,000, 1:1000 to 1:11,000, 1:1000 to 1:12,000, 1:1000 to 1:13,000, 1:100 to 1:14,000, 1:1000 to 1:15,000, 1:1000 to 1:16,000, 1:1000 to 1:17,000, 1:1000 to 1:18,000, 1:1000 to 1:19,000, or 1:1000 to 1:20,000.

In accordance with these embodiments, the methods and compositions of the present disclosure can be used to overcome hurdles of antibody-based therapeutics (e.g., immune globulin treatments). For example, compositions and methods of the disclosure overcome the risk of exacerbating COVID-19 severity via antibody-dependent enhancement (ADE). Although an understanding of a mechanism is not needed to practice the present disclosure, and while the disclosure is not limited to any particular mechanism, in one embodiment, the methods of compositions of the disclosure specifically identify and exclude plasma samples (e.g., prior to pooling plasma samples and immune globulin isolation) containing high levels of SARS-CoV-2 specific antibodies that lack neutralizing antibodies or that contain antibodies at sub-neutralizing levels that bind to viral antigens without blocking or clearing infection.

ADE has been documented to increase the severity of multiple viral infections, including other respiratory viruses such as respiratory syncytial virus (RSV) (see, e.g., Kim et al., Am. J. Epidemiol. 89, 422-434 (1969); and Graham, Vaccine 34, 3535-3541 (2016)) and measles (See, e.g., Nader et al., J. Pediatr. 72, 22-28 (1968); and Polack, Pediatr. Res. 62, 111-115 (2007)). ADE in respiratory infections is included in a broader category named enhanced respiratory disease (ERD), which also includes non-antibody-based mechanisms such as cytokine cascades and cell-mediated immunopathology. ADE caused by enhanced viral replication has been observed for other viruses that infect macrophages, including dengue virus and feline infectious peritonitis virus (FIPV).

ADE has been documented to occur through two distinct mechanisms in viral infections: by enhanced antibody-mediated virus uptake into Fc gamma receptor IIa (FcγRIIa)-expressing phagocytic cells leading to increased viral infection and replication, or by excessive antibody Fc-mediated effector functions or immune complex formation causing enhanced inflammation and immunopathology. Both ADE pathways can occur when non-neutralizing antibodies or antibodies at sub-neutralizing levels bind to viral antigens without blocking or clearing infection.

ADE can be measured in several ways, including in vitro assays (which are most common for the first mechanism involving FcγRIIa-mediated enhancement of infection in phagocytes), immunopathology or lung pathology. ADE via FcγRIIa-mediated endocytosis into phagocytic cells can be observed in vitro and has been extensively studied for macrophage-tropic viruses, including dengue virus in humans. In this mechanism, non-neutralizing antibodies bind to the viral surface and traffic virions directly to macrophages, which then internalize the virions and become productively infected. Since many antibodies against different dengue serotypes are cross-reactive but non-neutralizing, secondary infections with heterologous strains can result in increased viral replication and more severe disease, leading to major safety risks. Non-neutralizing antibodies, or antibodies at sub-neutralizing levels, enhanced entry into alveolar and peritoneal macrophages, which are thought to disseminate infection and worsen disease outcome.

Accordingly, while an understanding of a mechanism is not needed to practice the present disclosure, and while the disclosure is not limited to any particular mechanism, in one embodiment, the methods and compositions of the disclosure provide pooled plasma compositions and immune globulin prepared therefrom that contain high titers of SARS-CoV-2 neutralizing antibodies that prevent trafficking of virions to macrophages and infection of the macrophages, and/or that prevent secondary infections and/or that prevent viral entry into alveolar or peritoneal macrophages (e.g., thereby eliminating the risk of ADE or ERD).

In the second described ADE mechanism that is best exemplified by respiratory pathogens, Fc-mediated antibody effector functions can enhance respiratory disease by initiating a powerful immune cascade that results in observable lung pathology (See, e.g., Ye et al., Front. Immunol. 8, 317 (2017); and Winarski, et al., Proc. Natl Acad. Sci. USA 116, 15194-15199 (2019)). Fc-mediated activation of local and circulating innate immune cells such as monocytes, macrophages, neutrophils, dendritic cells and natural killer cells can lead to dysregulated immune activation despite their potential effectiveness at clearing virus-infected cells and debris. For non-macrophage tropic respiratory viruses such as RSV and measles, non-neutralizing antibodies have been shown to induce ADE and ERD by forming immune complexes that deposit into airway tissues and activate cytokine and complement pathways, resulting in inflammation, airway obstruction and, in severe cases, leading to acute respiratory distress syndrome.

Accordingly, while an understanding of a mechanism is not needed to practice the present disclosure, and while the disclosure is not limited to any particular mechanism, in one embodiment, the methods and compositions of the disclosure provide pooled plasma compositions and immune globulin prepared therefrom that contain high titers of SARS-CoV-2 neutralizing antibodies that prevent activation of local and circulating innate immune cells (e.g., monocytes, macrophages, neutrophils, dendritic cells and natural killer cells) and prevent dysregulated immune activation, prevent immune cascades that results in observable lung pathology, prevent and/or reduce ADE and ERD by blocking deposit of immune complexes in airway tissue, and/or prevent inflammation, airway obstruction and, acute respiratory distress syndrome.

### Immune Globulin Production

The disclosure further provides a method of producing an immune globulin comprising elevated levels of neutralizing antibody titers to one or more coronaviruses, which comprises (a) pooling plasma samples from a plurality of human plasma donors to produce a pooled plasma composition, and (b) detecting coronavirus neutralizing antibodies in the pooled plasma composition using the methods described above, wherein the coronavirus neutralizing antibody titer in the pooled plasma composition is from at least 40 to about 30,000. Descriptions of pooling plasma samples, generating a pooled plasma composition, and components thereof set forth above in connection with methods of detecting coronavirus neutralizing antibodies also are applicable to the method of producing an immune globulin.

In general, plasma samples taken from a plurality of individuals may be screened for antibodies against a particular antigen. Those samples which have certain antibody titers against the antigen are pooled in order to make an immune globulin preparation for treatment of infections caused by the particular antigen or an organism or virus containing such antigen (e.g., a coronavirus).

The screening of the plasma samples for antibodies is carried out by testing each of the plasma samples for the appropriate antibodies through the use of an appropriate assay, such as the assays and methods described herein. Other assays which may be employed include competitive assays, inhibition assays, immunofluorescence assays, enzyme-linked immunosorbent (ELISA) assays, sandwich assays, and neutralization assays. In determining antibody titers for each of the plasma samples, the same assay is carried out for each sample. Those samples having the desired antibody titers are then selected for the production of a pooled immunoglobulin preparation.

Embodiments of the present disclosure are not limited by the type of viral pathogens for which the pooled plasma comprises elevated levels of specific antibody titers. For example, the pooled plasma composition may comprise elevated levels of pathogen-specific antibody titers to one or more of coronavirus (e.g., coronavirus OC43, coronavirus 229E, coronavirus NL63, coronavirus HKU1, MERS-CoV, SARS-CoV, and/or SARS-CoV-2 (COVID-19)) and one or more other respiratory pathogens including, but not limited to, respiratory syncytial virus (RSV), influenza A virus, influenza B virus, parainfluenza virus type 1, parainfluenza virus type 2, metapneumovirus, S. pneumonia, H. influenza, L. pneumophila, and group A Streptococcus, or any other respiratory pathogen known in the art or described herein. In some embodiments, a pooled sample comprising higher neutralizing antibody titers against one virus also has proportionally higher neutralizing antibody titers against other pathogens. For example, pooled plasma samples can be obtained from a plurality of donor human subjects having increased antibody titers against a coronavirus (e.g., at least 1.2 fold greater than antibody titers from a corresponding control sample or an antibody neutralization titer of at least 40 to about 30,000), and these pooled plasma samples can also have proportionally increased antibody titers against at least a second pathogen, including, but not limited to, respiratory syncytial virus (RSV), influenza A virus, influenza B virus, parainfluenza virus type 1, parainfluenza virus type 2, metapneumovirus, coronavirus OC43, coronavirus 229E, coronavirus NL63, coronavirus HKU1, MERS-CoV, SARS-CoV, SARS-CoV-2 (COVID-19), S. pneumonia, H. influenza, L. pneumophila, and group A Streptococcus. Additionally, in some embodiments, pooled plasma samples can be obtained from a plurality of donor human subjects having increased antibody titers against a coronavirus (e.g., at least 1.2 fold greater than antibody titers from a corresponding control sample or an antibody neutralization titer of at least 1000 to 8000), and these pooled plasma samples can also have proportionally increased antibody titers against another respiratory virus, such as RSV (e.g., at least 1.2 fold greater than antibody titers from a corresponding control sample or an antibody neutralization titer of at least 40 to about 30,000). In one embodiment, immune globulin produced from the pooled plasma composition comprises a coronavirus-specific neutralizing antibody titer that is at least 1.2 fold greater (e.g., 1.2 fold, 1.3 fold, 1.4 fold, 1.5 fold, 1.6 fold, 1.7 fold, 1.8 fold, 1.9 fold, 2 fold, 3 fold, 4 fold, 5 fold 6 fold, 7 fold, 8 fold, 9 fold, 10 fold or more) than the corresponding antibody titer found in a mixture of plasma samples obtained from 100 or more random human subjects. In some embodiments, the antibody neutralization titer for a coronavirus is at least 40 to about 30,000.

Pooled plasma compositions can be used to prepare immune globulin (e.g., that is subsequently administered to a subject) via methods known in the art (e.g., fractionation, purification, isolation, etc.) and disclosed herein. Methods for preparing immune globulin from pooled plasma samples also is described in U.S. Pat. No. 10,683,343. The present disclosure provides that both pooled plasma compositions and immunoglobulin prepared from same may be administered to a subject to provide prophylactic and/or therapeutic benefits to the subject. Accordingly, the term pooled plasma composition may refer to immunoglobulin prepared from pooled plasma/pooled plasma samples.

In one embodiment, the pooled plasma composition and/or immunoglobulin provides a therapeutic benefit to a subject administered the composition that is not achievable via administration of a mixture of plasma samples obtained from a plurality of random human subjects and/or immunoglobulin prepared from same. Embodiments of the present disclosure are not limited by the type of therapeutic benefit provided. Indeed, a variety of therapeutic benefits may be attained including those described herein. In one embodiment, the pooled plasma and/or immunoglobulin possesses enhanced viral neutralization properties compared to a mixture of plasma samples obtained from a plurality of random human subjects or immunoglobulin prepared from same. For example, in one embodiment, the pooled plasma possesses enhanced viral neutralization properties against one or more (e.g., two, three, four, five or more) viral pathogens (e.g., respiratory pathogens). In a further embodiment, the enhanced viral neutralization properties reduce and/or prevent infection in a subject administered the composition for a duration of time that is longer than, and not achievable in, a subject administered a mixture of plasma samples obtained from a plurality of random human subjects. For example, in one embodiment, immunoglobulin prepared from pooled plasma according to the present disclosure (e.g., characterized, selected and blended according to the embodiments of the present disclosure) that is administered to a subject results in a significant, concentration dependent anti-coronavirus neutralization activity and/or other pathogen (e.g., RSV, influenza A virus, influenza B virus, parainfluenza virus type 1, parainfluenza virus type 2, metapneumovirus, coronavirus OC43, coronavirus 229E, coronavirus NL63, coronavirus HKU1, MERS-CoV, SARS-CoV, or SARS-CoV-2 (COVID-19), S. pneumonia, H. influenza, L. pneumophila, and group A Streptococcus) specific neutralization activity that is not achieved or achievable using immunoglobulin prepared from randomly pooled plasma samples (e.g., over a period of hours, days, weeks or longer). In one embodiment, the therapeutic benefit of a pooled plasma and/or immunoglobulin of the present disclosure is enhanced viral neutralization properties that reduce or prevent infection (e.g., coronavirus infection) in a subject administered the pooled plasma and/or immunoglobulin for a duration of time that is longer than, and not achievable in, a subject administered a mixture of pooled plasma and/or immunoglobulin prepared from same obtained from a plurality of random human subjects. In one embodiment, the therapeutic benefit is a significant reduction in viral load of a subject administered the pooled plasma and/or immunoglobulin compared to a control subject not receiving same. In a further embodiment, the pooled plasma and/or immunoglobulin significantly reduces lung histopathology in a subject administered the pooled plasma and/or immunoglobulin compared to a control subject not receiving same. In yet a further embodiment, the pooled plasma and/or immunoglobulin significantly reduces the level of pathogenic viral RNA in a tissue selected from lung, liver and kidney in a subject administered the pooled plasma and/or immunoglobulin compared to a control subject. In one embodiment, a subject administered immunoglobulin prepared from pooled plasma according to the present disclosure displays a mean fold increase in coronavirus neutralization titer that is at least 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 4.0, 5.0, 6.0, 7.0, 8.0 or more fold or more at a time point of at least 1-14 days (e.g., 14 day, 15 days, 16 days, 17 days, 18 days, 19 days or more) post administration of the immunoglobulin. In one embodiment, the pooled plasma and/or immunoglobulin prepared from same reduces the incidence of infection in a subject administered the composition. In another embodiment, a pooled plasma and/or immunoglobulin prepared from same reduces the number of days a subject administered the pooled plasma and/or immunoglobulin is required to be administered antibiotics (e.g., to treat infection). In yet another embodiment, a pooled plasma and/or immunoglobulin prepared from the same increases the trough level of circulating anti-respiratory pathogen specific antibodies in a subject (e.g., increases the level of neutralizing titers specific for viral pathogens, thereby providing protective levels of anti-respiratory pathogen specific antibodies between scheduled dates of administration of the pooled plasma and/or immunoglobulin prepared from same that are not maintained in a subject administered a mixture of plasma samples obtained from a plurality of more random human subjects (e.g., 50, 100, 200, 300, 400, 500 or more subjects) or immunoglobulin prepared from same).

### Kits

The disclosure provides a rapid detection kit for detecting coronavirus neutralizing antibodies, which comprises: (a) a solid support comprising a coronavirus cell receptor immobilized thereto; (b) a conjugate comprising a reporter molecule attached to a peptide comprising a receptor binding domain (RBD) of a coronavirus spike protein; (c) a positive control comprising panel of two or more coronavirus neutralizing monoclonal antibodies; and (d) a negative control comprising at least one coronavirus non-neutralizing antibody. A “rapid detection” kit (also referred to as a “point-of-care (POC)” kit) is a kit or device that can produce assay results in less than about an hour, and preferably in less than about 30 minutes. In some embodiments, a rapid detection kit can be used without the aid of additional equipment. A variety of rapid detection kits and systems, such as rapid immunoassays, are known in the art and can be adapted for use with the disclosed methods. For example, in some embodiments, the rapid detection kit may be a lateral flow kit, an ELISA kit, a latex agglutination kit, a flow-through kit, a biosensor, a lab-on-a-chip technology, or an optical immunoassay kit (see, e.g., Hesterberg, L. K and M. A. Crosby, Laboratory Medicine, 27(1): 41-46 (1996)). In some embodiments, the rapid detection kit is a lateral flow kit. A “lateral flow” kit employs a “lateral flow assay,” which is a paper-based platform for the detection and quantification of analytes in complex mixtures (see, e.g., Koczula, K. M. and Gallotta, A., Essays Biochem., 60(1): 111-120 (2016)). The sample is placed on a test device and the results are displayed within about 5 to about 30 minutes. The disclosure is not limited to any particular rapid detection kit or system, and other kits and systems used in the art for rapid detection of neutralizing antibodies may be used to perform the methods described herein. Descriptions of the solid support, conjugate, positive control, negative control, and components thereof set forth above in connection with methods of detecting coronavirus neutralizing antibodies also are applicable to the aforementioned kit.

In certain embodiments, the kit can comprise instructions for assaying a sample for coronavirus neutralizing antibodies using the methods described herein, e.g., an ELISA. The instructions can be in paper form or computer-readable form, such as a disk, CD, DVD, or the like. Alternatively or additionally, the kit can comprise a calibrator or control, e.g., purified, and optionally lyophilized, and/or at least one container (e.g., tube, microtiter plates or strips, which can be already coated with a coronavirus cell receptor) for conducting the assay, and/or a buffer, such as an assay buffer or a wash buffer, either one of which can be provided as a concentrated solution, a trigger solution for the detectable label (e.g., a chemiluminescent label), or a stop solution. Ideally, the kit comprises all components, i.e., reagents, standards, buffers, diluents, etc., which are necessary to perform the assay. The instructions also can include instructions for generating a standard curve or a reference standard for purposes of quantifying neutralizing antibodies.

The following examples further illustrate the disclosure but, of course, should not be construed as in any way limiting its scope.

### EXAMPLE 1

This example describes a serology assay for detecting of anti-SARS-CoV-2 receptor binding domain (RBD) antibodies.

Serum or plasma samples were added to wells of an ELISA micro plate coated with recombinant SARS-CoV-2 RBD. Anti-SARS-CoV-2 RBD antibodies present in the sample bind to the rSARS-CoV-2 RBD on the coated plate. Following an incubation period, unbound sample matrix was washed away. An HRP enzyme-labeled anti-human IgG, Fc-specific antibody conjugate was added to the plate. This conjugate specifically binds to the anti-RBD antibodies bound to the rRBD on the micro plate. Following an incubation period, unbound conjugate was washed away.

A colorimetric enzyme substrate was added to the plate. The HRP enzyme reacted with the substrate, and the resulting color change was proportional to the amount of anti-SARs-CoV-2 RBD antibodies bound to the plate. The assay Cutoff Control value was determined by screening a large number (>500) human plasma samples that were collected prior to the COVID-19 outbreak (Prior to Dec. 1, 2019). The cutoff selection was performed by estimating the mean of the negative specimens plus four times the standard deviation. The assay is used to determine the status of an unknown test sample by determining the average assay Cutoff Control value. This is followed by calculating the Sample Cutoff Ratio of the OD 450 nm obtained from the test sample divided by the OD 450 nm of the average Cutoff Control value. The negative cutoff was <0.8 (indicates no detectable IgG antibodies targeting the SARS-CoV-2 antigen). The positive cutoff was ≥1.2 (indicates the presence of detectable IgG antibodies targeting the SARS-CoV-2 antigen). A borderline value was 0.8≤>1.2 (indicates a definitive test result is not possible). The results are shown in Tables 1 and FIG. 2B. The scoring criteria based on adjusted absorbance values is shown in Table 2.

TABLE 1

| Sample ID | OD 450 | Score |
|---|---|---|
| 6207 | 0.2832 | + |
| 6208 | 2.0912 | +++ |
| 6211 | 1.0402 | ++ |
| 6215 | 0.4092 | + |
| 6217 | 2.3472 | +++ |
| 6228 | 0.9882 | ++ |
| 6232 | 2.3602 | +++ |
| 6233 | 2.5072 | ++++ |
| 6138 | 0.2032 | +/− |
| 2453 | −0.082 | − |
| 6001 | 0.1902 | +/− |

TABLE 2

| Adjusted Absorbance | Score |
|---|---|
| <0.00 | − |
| 0.0-0.25 | +/− |
| 0.25-0.5 | + |
| 0.5-1.5 | ++ |
| 1.5-2.5 | +++ |
| >2.5 | ++++ |

<0.00 = Actual absorbance − Negative Cutoff
0.00-0.25 = Actual absorbance falls between Negative and Positive Cutoff
>0.25 = Actual absorbance − Positive Cutoff

### EXAMPLE 2

This example demonstrates an assay that detects neutralizing anti-SARS-CoV-2 receptor binding domain (RBD) antibodies.

A COVID-19 micro-ELISA test for detecting neutralizing antibodies was designed for the semi-quantification of human anti-SARS-CoV-2 antibodies of all Ig classes present in human serum or plasma (Test Specimen). The test is a solid phase enzyme-linked immunosorbent assay (ELISA) using a chromogenic enzyme substrate as an indicator. The SARS-CoV-2 recombinant viral entry receptor protein, angiotensin-converting enzyme 2 (ACE2), is immobilized to polystyrene wells of a microplate (solid phase). In the wells of a separate incubation plate, test specimens, negative control and positive control are diluted and added to the wells along with the diluted soluble recombinant SARS-CoV-2 receptor binding domain (RBD) protein conjugated to horseradish peroxidase. During a shaking and incubation period, antibodies with specificity to SARS-CoV-2 RBD, if present in test specimens and controls, will bind to the RBD-horseradish peroxidase conjugate. After the incubation period, the controls and test specimens are transferred from the wells of the incubation plate to the wells of the test plate containing immobilized recombinant human ACE2 and allowed to incubate. After an incubation period, the wells are washed to remove unbound sample matrix and an enzyme substrate-chromogen (hydrogen peroxide, H₂O₂, and tetramethylbenzidine, TMB) is added to each well and incubated, resulting in the development of a blue color. The intensity of the blue color is indirectly proportional to the concentration of the SARS-CoV-2 neutralizing antibodies in the test specimens. An assay stop solution is added at the 20 minute mark post addition of enzyme substrate-chromogen and the color intensity is read in an absorbance plate reader at wavelength 450 nm.

Positive and negative quality controls are provided to ensure the integrity of the test. Specifically, a positive control value is generated using a known monoclonal antibody capable of neutralizing, in vivo and in vitro, the SARS-CoV-2 virus by blocking the binding of the SARS-CoV-2 receptor binding domain (RBD) to the human angiotensin-converting enzyme 2 (ACE2). Serum and plasma that test negative or positive for neutralization of the live SARS-CoV-2 virus have been shown to correlate with the results of this assay.

The COVID-19 micro-ELISA test then determines the status of an unknown test specimen/sample by determining the Sample Neutralization Index (SNI) for each sample using the following algorithm:

SNI=[1−(Sample OD₄₅₀ nm/Negative Control OD₄₅₀ nm)]/[1−(Positive Control OD₄₅₀ nm/Negative Control OD₄₅₀ nm)]

An SNI=1 indicates that the unknown test specimen has a % neutralization equal to the positive control. An SNI>1 indicates the unknown test specimen has a % neutralization greater than the positive control. An SNI<1 indicates the unknown test specimen has a % neutralization less than the positive control.

The specific assay procedure is as follows: (1) Allow all kit reagents to stand for 30 minutes to reach room temperature (18-30° C.) and gently mix each vial by vortexing on low speed or inverting 10 times. (2) The Positive Control, Negative Control and the Calibrator Control must be assayed in duplicate on the 96 well Microplate each time the test is performed. Up to ninety (90) test specimens may be ran in singlicate on each full plate. (3) Add 60 μl of the diluted RBD-enzyme conjugate into each well of the Incubation Plate. (4) Pipette 60 μl of the two step diluted Positive Control, Negative Control and Calibrator Control into the individual microwells of the Incubation Plate in duplicate. (5) Pipette 60 μl of the diluted Test Specimens into the corresponding individual microwells of the Incubation Plate in singlicate. (6) Place the Incubation Plate on a microplate shaker set at 300 rpm for 30 minutes at room temperature (18-30° C.). (7) Place sufficient microplate strip wells containing immobilized recombinant ACE2 in a strip holder to run all assay controls in duplicate and test specimens in singlicate. (8) Incubate the test plate for 30 minutes at +37° C.±1° C. in an incubator without carbon dioxide. For manual processing of microplate wells, cover the finished test plate with an adhesive protective plate sealer and start incubation. When using automated microplate processors, for incubation, follow the recommendations of the instrument manufacturer.

This assay was performed on serum or plasma samples, the results of which are shown in FIGS. 2A-2C. This assay and the assay described in Example 1 also were performed on 500 negative samples. 500 true negative results were obtained on both assays with this large cohort, indicating 100% specificity on a sample cohort, which is over six times more than what is required by the FDA. The FDA has revised the requirement for testing of positive samples on serology tests. The FDA requires a sampling of positive testing in categories of days past PCR testing (i.e., 0-7 days, 8-14-days and >14 days).

### EXAMPLE 3

This example describes the use of a COVID-19 micro-ELISA neutralizing antibody test in high throughput screening for SARS-CoV-2 neutralizing antibodies. A COVID-19 micro-ELISA neutralizing antibody test as described in Example 2 was used to differentiate neutralizing capacity of a panel of antibodies isolated from COVID-19 survivors. The results are shown in FIG. 3A. Clones A, B, C and D recognized the r-SARS-CoV-2 receptor binding domain (RBD) and clone E was specific for the r-N-terminal domain (NTD) of the spike protein. Antibodies were spiked in negative human plasma collected in 2020.

For comparison, a COVID-19 micro-ELISA serology assay as described in Example 1 also was performed on a panel of monoclonal antibodies isolated from COVID-19 survivors. Dose dependent binding curves from this assay are shown in FIG. 3B. Clones A, B, C and D recognized the r-SARS-CoV-2 receptor binding domain (RBD) and clone E was specific for the r-N-terminal domain (NTD) of the spike protein. When these antibody binding curves (FIG. 3B) were compared with their neutralization capacity using a neutralization assay as described in Example 2 (FIG. 3A), the results indicated that neutralization capacity does not directly correspond to RBD binding titer, and a neutralization assay as described in Example 2 is required to determine neutralizing capacity of monoclonal antibodies present in plasma or serum. For example, maximum % neutralization of Clone A is higher than that of Clones B and C (at 10 μg/ml), as shown in FIG. 3A. However, in FIG. 3B, Clones B and C exhibit higher RBD binding titers than that of Clone A. Therefore, the SARS-CoV-2 micro-ELISA neutralizing antibody test described herein can be used to determine % neutralization capacity for any given sample based on a positive control(s) (e.g., coronavirus neutralizing monoclonal antibodies), as opposed to simply identifying SARS-CoV-2 binding capacity. The SARS-CoV-2 micro-ELISA neutralizing antibody test described herein has the added benefit of not requiring the use of live viruses.

### EXAMPLE 4

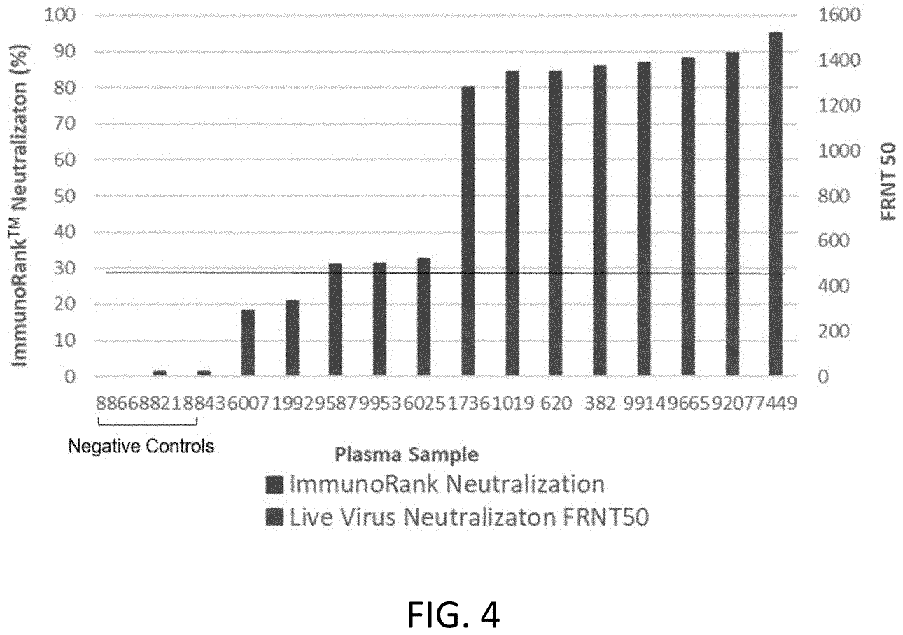

A COVID-19 neutralization micro-ELISA assay as described in Example 2 was compared to a live virus focus reduction neutralization test (FRNT50), and the results are shown in FIG. 4. Samples exhibiting >80% neutralization in the assay described herein showed FRNT50 values of approximately 300 or greater, as shown in Table 3 and FIG. 4.

TABLE 3

| | | % | Serology |
|---|---|---|---|
| | FRNT 50 | Neutralization | Rank |
| 8843 | 0 | 1.40 | − |
| 8821 | 0 | 1.20 | − |
| 8866 | 0 | 0.00 | − |
| 9587 | 29.54 | 30.90 | 2+ |
| 1992 | 35.69 | 21.00 | 2+ |
| 9953 | 41.98 | 31.40 | 2+ |
| 6025 | 68.73 | 32.40 | 2+ |
| 6007 | 201.94 | 18.10 | 2+ |
| 1736 | 294.64 | 80.20 | 3+ |
| 382 | 333.67 | 85.80 | 3+ |
| 1019 | 372.3 | 84.30 | 3+ |
| 620 | 446.63 | 84.30 | 3+ |
| 9914 | 540.83 | 86.80 | 3+ |
| 9207 | 718.39 | 89.70 | 3+ |
| 9665 | 1047.01 | 88.00 | 3+ |
| 7449 | >1500 | 95.10 | 4+ |

### EXAMPLE 5

Results produced by the COVID-19 neutralization ELISA as described in Example 2 were compared to results produced the COVID-19 serology ELISA as described in Example 1. Each assay was performed on 100 convalescent plasma units obtained from the New York Blood Center. The results are presented in Tables 4 and 5.

TABLE 4
Comparison of Serology Results to Neutralization Results

| | | Sample Neutralization |
|---|---|---|
| Sample Cutoff Ratio (SCR) | | Index (SNI %) |
| From Trace IgG Serology | n= | Positives ≥ 25% SNI |
| <1.2 (Indicates Negative) | 23 | 1 |
| 1.2-3.6 | 38 | 10 |
| 3.7-4.8 | 21 | 19 |
| 4.9-6.1 | 18 | 17 |

TABLE 5
Comparison of Neutralization Results to Serology Results

| Sample Neutralization | | |
|---|---|---|
| Index SNI % | n= | Average SCR |
| <25% | 53 | 1.5 |
| 25-50% | 19 | 3.6 |
| 51-75% | 17 | 4.7 |
| >75% | 11 | 5.1 |

### EXAMPLE 6

This example compares the results that were obtained using the ELISA as described in Example 2 to the results from the focus reduction neutralization test (FRNT) for positive percent agreement (PPA) with 150 positive samples, and negative percent agreement (NPA) with negative samples (95% confidence interval [CI] was calculated using the Wilson Method). Cross-reactivity of antibodies was analyzed with this ELISA to 11 viruses including the common coronaviruses NL63, 2293, OC43 and HKU1.

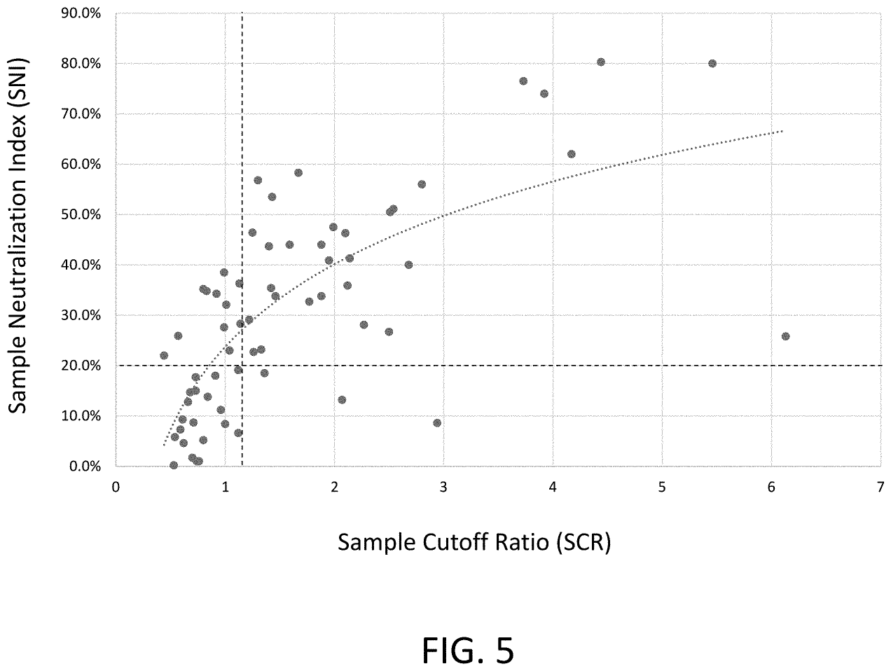

Results demonstrated that when comparing the ELISA data and the FRNT PPA for the 150 samples, there was a very close correlation between the extent of the neutralization in these two assays. ELISA specificity was demonstrated, as there was no cross-reactivity with other viruses measured in the 55 samples tested. In addition, of the 531 negative plasma samples collected from healthy donors prior to the COVID outbreak, 527 samples were negative for neutralizing antibodies resulting in 99.3% specificity. Furthermore, when screening 100 convalescent plasma donor samples, only 61% of the samples contained SARS-CoV-2 neutralizing antibodies (Table 6). About 80% of these exhibited low to moderate neutralization and only 20% contained high neutralization activity. Additionally, as shown in FIG. 5, the % coronavirus neutralization (SNI) is independent of amount of binding (SCR) detected. For example, data points in the upper left of the graph (above the trendline) are samples that have high neutralization (50%-60%) but low binding, and data points below the trendline are samples with high binding (>2) but low % neutralization (>30%).

Thus, data obtained using the SARS-CoV-2 ELISA neutralization assay as described in Example 2 not only correlates with FRNT data, but unlike FRNT and other currently used plaque reduction cell-based assays, the SARS-CoV-2 neutralization assay described herein does not require the use of live viruses, which significantly reduces the time, cost, and resources for determining accurate antibody neutralization titers in a sample.

TABLE 6
Representative % neutralization (SNI) data and binding
capacity (SCR) data from plasma samples.

| Sample ID | SCR | SNI |
|---|---|---|
| W04702003087300 | 0.61 | 0.0% |
| W04702003091300 | 0.62 | 0.0% |
| W04702003096200 | 1.25 | 46.4% |
| W04702003096300 | 1.12 | 6.6% |
| W04702009742500 | 1.46 | 33.8% |
| W04702009746100 | 0.42 | 0.0% |
| W04702010678200 | 0.83 | 34.8% |
| W04702010683800 | 0.44 | 22.0% |
| W04702010684000 | 0.92 | 34.3% |
| W04702011215200 | 1.26 | 22.7% |
| W04702011218100 | 2.07 | 13.2% |
| W04702011219400 | 1.88 | 44.0% |
| W04702011221400 | 0.33 | 0.0% |
| W04702011222400 | 0.09 | 0.0% |
| W04702011224000 | 2.1 | 46.3% |
| W04702011263800 | 0.34 | 0.0% |
| W04702011263900 | 3.92 | 74.0% |
| W04702011264300 | 0.61 | 0.0% |
| W04702011270900 | 0.48 | 0.0% |
| W04702011401900 | 6.13 | 25.8% |
| W04702011404500 | 1.99 | 47.5% |
| W04702011407400 | 2.8 | 56.0% |
| W04702011413600 | 1.95 | 40.9% |
| W04702011415500 | 2.68 | 40.0% |
| W04702011444400 | 0.73 | 0.0% |
| W04702011446100 | 4.17 | 62.0% |
| W04702011462900 | 0.85 | 0.0% |
| W04702011464700 | 2.27 | 28.1% |
| W04702011473000 | 0.7 | 1.7% |
| W04702011523200 | 0.91 | 18.0% |
| W04702011623700 | 2.12 | 35.9% |
| W04702011677800 | 1.77 | 32.7% |
| W04702011689100 | 0.59 | 7.3% |
| W04702011689700 | 1.04 | 23.0% |
| W04702011695400 | 3.73 | 76.5% |
| W04702011786300 | 0.99 | 27.6% |
| W04702011796700 | 0.53 | 0.2% |
| W04702011810800 | 2.51 | 50.5% |
| W04702011810900 | 0.99 | 38.5% |
| W04702011812700 | 0.73 | 0.0% |
| W04702011896400 | 1.43 | 53.5% |
| W04702011945700 | 0.29 | 0.0% |
| W04702011947100 | 0.72 | 0.0% |
| W04702011947800 | 1.33 | 23.2% |
| W04702012001600 | 0.84 | 13.8% |
| W04702012006300 | 0.6 | 0.0% |
| W04702012103000 | 0.61 | 9.3% |
| W04702012104200 | 1.4 | 43.7% |
| W04702012104600 | 1.3 | 56.8% |
| W04702012105700 | 0.36 | 0.0% |
| W04702012326400 | 1.13 | 36.3% |
| W04702012331000 | 0.57 | 25.9% |
| W04702012410200 | 1.67 | 58.3% |
| W04702012466000 | 1.14 | 28.3% |
| W04702012469400 | 0.96 | 11.2% |
| W04702012533200 | 1.01 | 32.1% |
| W04702012698500 | 0.73 | 15.0% |
| W04702012782000 | 0.52 | 0.0% |
| W04702012785900 | 0.8 | 35.2% |
| W04702012803900 | 0.5 | 0.0% |
| W04702012880100 | 0.54 | 5.8% |
| W04702012881700 | 1.88 | 33.8% |
| W04702012885300 | 2.42 | 0.0% |
| W04702012886800 | 0.71 | 8.7% |
| W04702013110000 | 0.47 | 0.0% |
| W04702013280500 | 2.54 | 51.1% |
| W04702013289700 | 0.55 | 0.0% |
| W04702013448700 | 0.47 | 0.0% |
| W04702013475700 | 0.74 | 1.0% |
| W04702013475900 | 5.46 | 80.0% |
| W04702013528300 | 0.66 | 12.8% |
| W04702013641700 | 2.5 | 26.7% |
| W04702013643100 | 0.73 | 17.7% |
| W04702013644400 | 1.00 | 8.4% |
| W04702013733000 | 0.62 | 4.6% |
| W04702013775200 | 0.54 | 0.0% |
| W04702013792900 | 1.22 | 29.1% |
| W04702014044800 | 0.8 | 5.2% |
| W04702014058700 | 0.54 | 0.0% |
| W04702014066900 | 0.9 | 0.0% |
| W04702014189900 | 0.84 | 0.0% |
| W04702014198200 | 0.78 | 0.0% |
| W04702014257400 | 4.44 | 80.3% |
| W04702014381600 | 1.59 | 44.0% |
| W04702014502300 | 0.49 | 0.0% |
| W04702014516200 | 0.52 | 0.0% |
| W04702014536000 | 0.4 | 0.0% |
| W04702014542400 | 0.64 | 0.0% |
| W04702014601600 | 2.94 | 8.6% |
| W04702014624500 | 0.48 | 0.0% |
| W04702015302100 | 0.51 | 0.0% |
| W04702015303400 | 0.37 | 0.0% |
| W04702015304400 | 0.55 | 0.0% |
| W04702015316800 | 1.36 | 18.5% |
| W04702015330000 | 1.12 | 19.1% |
| W04702015334700 | 0.41 | 0.0% |
| W04702015364100 | 2.14 | 41.3% |
| W04702015486000 | 0.68 | 14.7% |
| W04702015486600 | 1.42 | 35.4% |
| W0470201573 8700 | 0.76 | 1.0% |

Trace Sample Cutoff Ratio (SCR): Positive ≥1.2; Negative <0.8; Borderline 0.8≤>1.2. ImmunoRank SNI: Positive ≥20%; Negative <20%.

Additionally, as shown below in Table 7, similar results were obtained using the ELISA as described in Example 2 and a plaque reduction neutralization test (PRNT). Results demonstrated that when comparing the ELISA data and the PRNT PPA for the samples listed, there was a very close correlation between the extent of the neutralization in these two assays (e.g., compare ImmunoRank SNI/Result to Average PRNT/Result).

TABLE 7
Representative % neutralization (SNI) data compared
to PRNT results from plasma/serum samples.

| | Sample | Days Post | ImmunoRank | Average |
|---|---|---|---|---|
| Sample ID | Type | Symptom Onset | SNI/Result | PRNT/Result |
| 1030007099 | Plasma | Pre Covid | 0%/negative | <128/negative |
| 1030007111 | Plasma | Pre Covid | 0%/negative | <128/negative |
| 4350 | Plasma | 31 | 55.7%/positive | 168/positive |
| 1030007340 | Plasma | Pre Covid | 3.5%/negative | <128/negative |
| 1030007344 | Plasma | Pre Covid | 0%/negative | <128/negative |
| 1030007548 | Plasma | Pre Covid | 3.7%/negative | <128/negative |
| 1682963a_1_7 | Serum | 6 | 68.1%/positive | >4096/positive |
| 1030000128 | Plasma | Pre Covid | 0%/negative | <128/negative |
| 1030000166 | Plasma | Pre Covid | 0%/negative | <128/negative |
| 4156 | Plasma | 21 | 38.5%/positive | 693/positive |
| 1030000218 | Plasma | Pre Covid | 0%/negative | <128/negative |
| 1030000355 | Plasma | Pre Covid | 0%/negative | <128/negative |
| 1030000362 | Plasma | Pre Covid | 0%/negative | <128/negative |
| 4331 | Plasma | 21 | 23.6%/positive | 337/positive |
| 1030000376 | Plasma | Pre Covid | 0%/negative | <128/negative |
| 1030000390 | Plasma | Pre Covid | 0%/negative | <128/negative |
| 1030000392 | Plasma | Pre Covid | 0%/negative | <128/negative |
| 4326 | Plasma | 26 | 57.7%/positive | 884/positive |
| 1030000399 | Plasma | Pre Covid | 0%/negative | <128/negative |
| 1030000618 | Plasma | Pre Covid | 0%/negative | <128/negative |
| 1687022a_1_7 | Serum | 26 | 72.1%/positive | 1413/positive |
| 1690862a_1_7 | Serum | 16 | 83%/positive | 3589/positive |
| 1030002382 | Plasma | Pre Covid | 0%/negative | <128/negative |
| 1030002679 | Plasma | Pre Covid | 0%/negative | <128/negative |
| 1030002699 | Plasma | Pre Covid | 0%/negative | <128/negative |
| 4153 | Plasma | 35 | 39.8%/positive | 375/positive |
| 1030005053 | Plasma | Pre Covid | 0%/negative | <128/negative |
| 1030005648 | Plasma | Pre Covid | 1.8%/negative | <128/negative |
| 1030004220 | Plasma | Pre Covid | 3.9%/negative | <128/negative |
| 1030005610 | Plasma | Pre Covid | 3.3%/negative | <128/negative |
| 1030005863 | Plasma | Pre Covid | 12.9%/negative | <128/negative |
| 4134 | Plasma | 21 | 28.6%/positive | 1151/positive |
| 1030006616 | Plasma | Pre Covid | 0%/negative | <128/negative |
| 1030006696 | Plasma | Pre Covid | 0%/negative | <128/negative |
| 4102 | Plasma | 17 | 30.2%/positive | 272/positive |
| 1030006714 | Plasma | Pre Covid | 0%/negative | <128/negative |
| 1030006850 | Plasma | Pre Covid | 0%/negative | <128/negative |
| 1030006853 | Plasma | Pre Covid | 0%/negative | <128/negative |
| 4332 | Plasma | 31 | 80.6%/positive | 3909/positive |
| 1030006855 | Plasma | Pre Covid | 0%/negative | <128/negative |
| 1030006925 | Plasma | Pre Covid | 0%/negative | <128/negative |
| 4184 | Plasma | 32 | 24.7%/positive | 1180/positive |
| 1030007007 | Plasma | Pre Covid | 0%/negative | <128/negative |
| 1030007032 | Plasma | Pre Covid | 0%/negative | <128/negative |
| 4112 | Plasma | 32 | 44.5%/positive | 152/positive |
| 1030002375 | Plasma | Pre Covid | 1.4%/negative | <128/negative |
| 1030002436 | Plasma | Pre Covid | 0%/negative | <128/negative |
| 1030002509 | Plasma | Pre Covid | 0%/negative | <128/negative |
| 4301 | Plasma | | 27.6%/positive | 1009/positive |
| 1030002604 | Plasma | Pre Covid | 0%/negative | <128/negative |
| 1030002666 | Plasma | Pre Covid | 0%/negative | <128/negative |
| 1030002866 | Plasma | Pre Covid | 0%/negative | <128/negative |
| 1685842a_1_6 | Serum | 15 | 98.9%/positive | 3838/positive |
| 1030003020 | Plasma | Pre Covid | 0%/negative | <128/negative |
| 1030003038 | Plasma | Pre Covid | 0%/negative | <128/negative |
| 1682442a_1_7 | Serum | 16 | 82%/positive | 2988/positive |
| 1030003118 | Plasma | Pre Covid | 0%/negative | <128/negative |
| 1030003882 | Plasma | Pre Covid | 0%/negative | <128/negative |
| 1030003923 | Plasma | Pre Covid | 0%/negative | <128/negative |
| 1030003947 | Plasma | Pre Covid | 0%/negative | <128/negative |
| 1030004191 | Plasma | Pre Covid | 0%/negative | <128/negative |
| 1030004468 | Plasma | Pre Covid | 0%/negative | <128/negative |
| 1030004248 | Plasma | Pre Covid | 0.1%/negative | <128/negative |
| 1030004666 | Plasma | Pre Covid | 0%/negative | <128/negative |
| 1030000898 | Plasma | Pre Covid | 0%/negative | <128/negative |
| 1683200a_1_7 | Serum | 21 | 76.9%/positive | 2631/positive |
| 1030000922 | Plasma | Pre Covid | 1%/negative | <128/negative |
| 1030000948 | Plasma | Pre Covid | 13.1%/negative | <128/negative |
| 1683999a_2_1 | Serum | 24 | 80.5%/positive | 2426/positive |
| 1030001036 | Plasma | Pre Covid | 18.2%/negative | <128/negative |
| 1030001288 | Plasma | Pre Covid | 5.2%/negative | <128/negative |
| 1030001334 | Plasma | Pre Covid | 12.4%/negative | <128/negative |
| 1030001650 | Plasma | Pre Covid | 9.3%/negative | <128/negative |
| 1030001733 | Plasma | Pre Covid | 0%/negative | <128/negative |
| 1030002081 | Plasma | Pre Covid | 1%/negative | <128/negative |
| 1030002853 | Plasma | Pre Covid | 0%/negative | <128/negative |
| 1030004312 | Plasma | Pre Covid | 0%/negative | <128/negative |
| 1030005687 | Plasma | Pre Covid | 0%/negative | <128/negative |
| 1030006151 | Plasma | Pre Covid | 4%/negative | >4096/positive |
| 1692691a_1_7 | Serum | 22 | 31.3%/positive | 1552/positive |
| 1030006225 | Plasma | Pre Covid | 2%/negative | <128/negative |
| 1030002994 | Plasma | Pre Covid | 8.1%/negative | <128/negative |
| 1684446a_1_7 | Serum | 23 | 89.3%/positive | >4096/positive |
| 1030005683 | Plasma | Pre Covid | 9.1%/negative | <128/negative |
| 1030005671 | Plasma | Pre Covid | 13.7%/negative | <128/negative |
| 4350 | Plasma | 31 | 50.4%/positive | 1056/positive |
| 1030004602 | Plasma | Pre Covid | 12.3%/negative | <128/negative |
| 1030005709 | Plasma | Pre Covid | 2.7%/negative | <128/negative |
| 1030004500 | Plasma | Pre Covid | 0%/negative | <128/negative |
| 1030003153 | Plasma | Pre Covid | 0%/negative | <128/negative |
| 1030004495 | Plasma | Pre Covid | 0%/negative | >4096/positive |
| 1030005708 | Plasma | Pre Covid | 0%/negative | <128/negative |
| 1030005749 | Plasma | Pre Covid | 0%/negative | <128/negative |
| 1030003277 | Plasma | Pre Covid | 0%/negative | <128/negative |
| 1030004231 | Plasma | Pre Covid | 0%/negative | <128/negative |
| 1030005532 | Plasma | Pre Covid | 0%/negative | <128/negative |
| 1691662a_1_6 | Serum | 24 | 90.4%/positive | >4096/positive |
| 1686294a_1_7 | Serum | 16 | 32.3%/positive | 1323/positive |
| 1682837a_1_7 | Serum | 25 | 49.8%/positive | 3418/positive |
| 1682512a_2_1 | Serum | 18 | 31.7%/positive | >4096/positive |
| 1682895a_2_1 | Serum | 2 | 73%/positive | 812/positive |
| 1687117a_1_6 | Serum | 41 | 92.8%/positive | >4096/positive |
| 1686592a_1_5 | Serum | 51 | 25.5%/positive | 3720/positive |
| 1686979a_1_5 | Serum | 30 | 88.3%/positive | >4096/positive |
| 1685836a_1_7 | Serum | 28 | 77%/positive | >4096/positive |
| 1684290a_2_2 | Serum | 35 | 69.4%/positive | >4096/positive |
| 1684072a_1_7 | Serum | 40 | 73.9%/positive | 3888/positive |

Days Post Symptom Onset: Days symptoms were reported by participant to time of collection.
ImmunoRank SNI/Result: Sample Neutralization Index (SNI) as calculated in accordance with the methods of the present disclosure, and interpretation of specimen status result.
Average PRNT/Result: Average PRNT TCID50 titer for each sample (done in triplicate).
PRNT Result: Negative = Titer < 128, Positive = Titer > 128.

### EXAMPLE 7

The data provided herein demonstrate that the coronavirus binding capacity and % neutralization capacity of any given sample are independent and not necessarily correlative. Given this, control samples can be generated for use in the assays of the present disclosure that enable a semi-quantitative determination of % neutralization. For example, in the assays described in Example 2, the positive control sample used corresponded to a sample determined to have a high coronavirus titer (i.e., a sample with an SNI of 20% and over was determined to be positive for coronavirus neutralizing antibodies). However, using other control samples (e.g., a sample with a medium coronavirus titer and/or a sample with a low coronavirus titer), a semi-quantitative determination of % neutralization of a test sample, as compared to the control samples, can be obtained.

As shown below in Table 8, % neutralization (SNI) was determined using the SARS-CoV-2 ELISA neutralization assay as described in Example 2 for various test samples.

TABLE 8
Representative % neutralization data from plasma samples.

| Sample ID | SNI % |
|---|---|
| 204799717 | 58.1% |
| 204799729 | 101.9% |
| 204799780 | 102.1% |
| 204799983 | 102.0% |
| 204800073 | 102.0% |
| 204800082 | 102.1% |
| 204800087 | 101.0% |
| 204922415 | 99.2% |
| 204922451 | 100.7% |
| 204922457 | 100.4% |
| 204922470 | 101.1% |
| 204925543 | 102.2% |
| 204925778 | 101.8% |
| 204925785 | 101.0% |
| 204926019 | 99.7% |
| 204926050 | 100.4% |
| 204926065 | 10.1% |
| 204926109 | 101.6% |
| 204926136 | 60.2% |
| 204927295 | 100.9% |
| 204927305 | 102.1% |
| 204927313 | 102.1% |
| 204975739 | 102.2% |
| 204975946 | 101.7% |
| 204976214 | 89.4% |
| 204976243 | 101.9% |
| 204977366 | 102.3% |
| 204977436 | 102.0% |
| 204980997 | 101.8% |
| 204981106 | 101.3% |
| 204981157 | 98.5% |
| 204981244 | 57.2% |
| 204981261 | 100.9% |
| 205052571 | 86.2% |
| 205052595 | 99.5% |
| 205053423 | 98.4% |
| 205053485 | 101.9% |
| 205125510 | 22.6% |
| 205125531 | 99.4% |
| 205125566 | 78.1% |
| 205125579 | 99.5% |
| 205125608 | 78.8% |
| 205125621 | 20.7% |
| 205125657 | 101.8% |
| 205126087 | 55.2% |
| 205126119 | 101.9% |
| 205126134 | 102.0% |
| 205126258 | 98.3% |
| 205126266 | 102.2% |
| 205126291 | 98.8% |
| 205126477 | 99.8% |
| 205126572 | 87.2% |
| 205126584 | 88.1% |
| 205126649 | 40.9% |
| 205126930 | 90.0% |

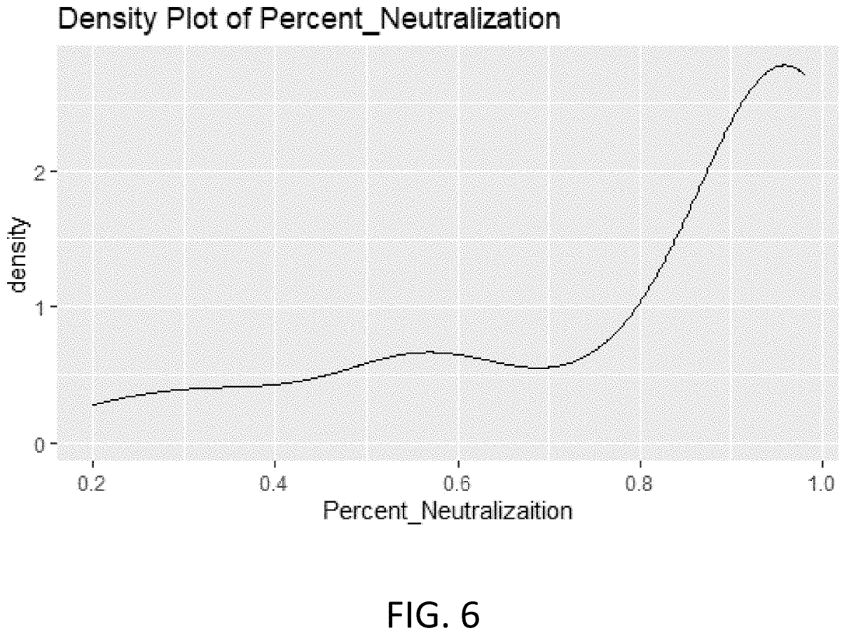

Using these data, a kernel density plot was created (FIG. 6). The kernel used was Gaussian. The plot was then examined to determine reasonable groupings and cutoff points for high, medium, and low effectiveness. As shown in FIG. 6, a value designated as a “low positive” was assigned a range between 0.2 and 0.4; a value designated as a “medium positive” was assigned a range between 0.4 and 0.7, and a value designated as a “high positive” was assigned a range between 0.7 and 1. Thus, a test sample with a % neutralization capacity in one of these ranges can be determined to have low, medium, or high coronavirus neutralization capacity.

### EXAMPLE 8

Another means for making a quantitative assessment of neutralization capacity of a test sample (e.g., serum or plasma) using the assays described in Example 2 involves making a series of dilutions of the sample and determining % neutralization (at a certain cutoff value). For example, as demonstrated below in Table 9, a cutoff value was set at 20% (e.g., a sample with an SNI of 20% and over was determined to be positive for coronavirus neutralizing antibodies), and the sample serially diluted (e.g., 1:10, 1:80, 1:320, and 1:1280). The % SNI was then determined for each of these dilutions using the neutralization assays of the present disclosure.

As shown in Table 9, all but one vaccinated subject had a positive % SNI at the lowest dilution (i.e., 1:10). However, even among vaccinated subjects with a positive % SNI, there was significant variance in the % SNI, which indicates that the % neutralization capacity among these subject may also be variable. To investigate this, the samples were tested at additional dilutions to determine % SNI. As shown in Table 9, only a subset of vaccinated subjects with a positive % SNI remained positive as the samples were diluted downward (1:80, 1:320, and 1:1280), and the % SNI value at the lowest dilution for a given subject was not necessarily predictive of the corresponding % SNI at the higher dilutions for that subject (e.g., compare subject 204981106 with subject 204981261, or subject 204925778 with subject 204925543). This highlights the variability in neutralization capacity among individuals and also demonstrates the utility of an assay that can quantitatively determine coronavirus % neutralization capacity.

TABLE 9
Representative quantification of coronavirus
% neutralization among vaccinated subjects.

| | Days Post | | | | |
|---|---|---|---|---|---|
| Sample ID | Vaccine | 1:10 | 1:80 | 1:320 | 1:1280 |
| 204925543 | 14 | 97.1% | 73.5% | 22.2% | 0.0% |
| 204925778 | 14 | 93.3% | 29.4% | 0.0% | 0.0% |
| 204925785 | 14 | 95.2% | 45.9% | 5.8% | 0.0% |
| 204975739 | 14 | 97.5% | 66.7% | 15.2% | 0.0% |
| 204975946 | 14 | 95.9% | 35.2% | 5.2% | 0.0% |
| 204976214 | 14 | 65.3% | 0.0% | 0.0% | 0.0% |
| 204976243 | 14 | 96.0% | 66.2% | 19.2% | 0.0% |
| 204977366 | 14 | 97.5% | 88.8% | 50.3% | 4.2% |
| 204977436 | 15 | 97.2% | 78.2% | 30.2% | 0.0% |
| 204926050 | 16 | 91.8% | 28.8% | 0.0% | 0.0% |
| 205052571 | 83 | 66.2% | 0.0% | 0.0% | 0.0% |
| 205052595 | 83 | 81.3% | 18.2% | 0.0% | 0.0% |
| 205053423 | 83 | 65.4% | 0.0% | 0.0% | 0.0% |
| 204981157 | 84 | 85.7% | 0.2% | 0.0% | 0.0% |
| 204981244 | 84 | 17.6% | 0.0% | 0.0% | 0.0% |
| 204980997 | 88 | 96.7% | 53.2% | 7.1% | 0.0% |
| 204981106 | 90 | 96.1% | 43.8% | 2.2% | 0.0% |
| 204981261 | 90 | 94.0% | 17.4% | 0.0% | 0.0% |
| 205125579 | 91 | 93.5% | 38.6% | 0.0% | 0.0% |

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and “at least one” and similar referents in the context of describing the disclosure (especially in the context of the following claims) are to be construed to cover both the singular and the plural, unless otherwise indicated herein or clearly contradicted by context. The use of the term “at least one” followed by a list of one or more items (for example, “at least one of A and B”) is to be construed to mean one item selected from the listed items (A or B) or any combination of two or more of the listed items (A and B), unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the disclosure and does not pose a limitation on the scope of the disclosure unless otherwise claimed. No language in the specification should be construed as indicating any non-claimed element as essential to the practice of the disclosure.

Preferred embodiments of this disclosure are described herein, including the best mode known for carrying out the disclosure. Variations of those preferred embodiments may become apparent to those of ordinary skill in the art upon reading the foregoing description. The inventors expect Skilled artisans may employ such variations as appropriate, and the disclosure is intended to be practiced otherwise than as specifically described herein. Accordingly, this disclosure includes all modifications and equivalents of the subject matter recited in the claims appended hereto as permitted by applicable law. Moreover, any combination of the above-described elements in all possible variations thereof is encompassed by the disclosure unless otherwise indicated herein or otherwise clearly contradicted by context.

1. A method of detecting coronavirus neutralizing antibodies in a sample, which method comprises:
(a) contacting a sample with a solid support comprising a coronavirus cell receptor immobilized thereto to form a mixture;
(b) contacting the mixture with a conjugate comprising a reporter molecule attached to a peptide comprising a receptor binding domain (RBD) of a coronavirus spike protein, whereby, if coronavirus neutralizing antibodies are present in the sample, the RBD binds to the coronavirus neutralizing antibodies and does not bind to the immobilized coronavirus cell receptor;
(c) detecting and quantifying a signal from the reporter molecule, wherein the amount of detected signal is inversely proportional to the amount of coronavirus neutralizing antibodies present in the sample; and
(d) performing steps (a)-(c) on a positive control comprising a panel of one or more coronavirus neutralizing monoclonal antibodies instead of the sample, and comparing the quantified signal of the positive control to the quantified signal of the sample to determine coronavirus neutralizing antibody capacity of the sample.

2. A method of detecting coronavirus neutralizing antibodies in a sample, which method comprises:
(a) contacting a sample with a conjugate comprising a reporter molecule attached to a peptide comprising a receptor binding domain (RBD) of a coronavirus spike protein to form a mixture, wherein the RBD binds to coronavirus neutralizing antibodies if present in the sample;
(b) contacting the mixture with a solid support comprising a coronavirus cell receptor immobilized thereto, whereby, if coronavirus neutralizing antibodies are present in the sample, the RBD bound to the coronavirus neutralizing antibodies does not bind to the immobilized coronavirus cell receptor;
(c) detecting and quantifying a signal from the reporter molecule, wherein the amount of detected signal is inversely proportional to the amount of coronavirus neutralizing antibodies present in the sample; and
(d) performing steps (a)-(c) on a positive control comprising a panel of one or more coronavirus neutralizing monoclonal antibodies instead of the sample, and comparing the quantified signal of the positive control to the quantified signal of the sample to determine coronavirus neutralizing antibody capacity of the sample.

3-4. (canceled)

5. The method of claim 1, wherein the coronavirus is coronavirus OC43, coronavirus 229E, coronavirus NL63, coronavirus HKU1, MERS-CoV, SARS-CoV, or SARS-CoV-2 (COVID-19).

6. The method of claim 5, wherein the coronavirus is SARS-CoV-2 (COVID-19).

7. The method of claim 1, wherein the coronavirus cell receptor is an angiotensin converting enzyme 2 (ACE2) receptor.

8. The method of claim 1, wherein the panel comprises three or more coronavirus neutralizing antibodies.

9-10. (canceled)

11. The method of claim 1, wherein the solid support is selected from a polystyrene micro-titer plate, a multiplexing chip array, polystyrene bead particles, magnetic particles, a cellulose membrane, and microparticles.

12. The method of claim 1, wherein the reporter molecule is an enzyme or a detectable tag.

13. The method of claim 12, wherein the reporter molecule is an enzyme selected from horseradish peroxidase (HRP) and alkaline phosphatase.

14. The method of claim 12, wherein the reporter molecule is a fluorescent tag.

15. (canceled)

16. The method of claim 1, wherein the sample comprises plasma, serum, or cell culture fluid.

17. The method of claim 1, wherein the sample comprises a pooled plasma composition comprising plasma samples from a plurality of human plasma donors.

18. The method of claim 17, wherein the plurality of human plasma donors is 100 or more.

19. The method of claim 17, wherein one or more of the plurality of human plasma donors have been clinically diagnosed with infection by the coronavirus and have recovered from the infection.

20. The method of claim 19, wherein one or more of the plurality of human plasma donors have been clinically diagnosed with an infection from at least a second pathogen and have recovered from the infection.

21. The method of claim 20, wherein the at least second pathogen is selected from respiratory syncytial virus (RSV), influenza A virus, influenza B virus, parainfluenza virus type 1, parainfluenza virus type 2, metapneumovirus, coronavirus OC43, coronavirus 229E, coronavirus NL63, coronavirus HKU1, MERS-CoV, SARS-CoV, SARS-CoV-2 (COVID-19), S. pneumonia, H. influenza, L. pneumophila, and group A Streptococcus.

22-23. (canceled)

24. The method of claim 17, wherein one or more of the plurality of human donors have been selected based on at least one pre-preselection criterion.

25. The method of claim 17, wherein one or more of the plurality of plasma donors have been vaccinated with a vaccine specific for the coronavirus.

26-42. (canceled)

43. A rapid detection kit for detecting coronavirus neutralizing antibodies, which comprises:
(a) a solid support comprising a coronavirus cell receptor immobilized thereto;
(b) a conjugate comprising a reporter molecule attached to a peptide comprising a receptor binding domain (RBD) of a coronavirus spike protein;
(c) a positive control comprising panel of two or more coronavirus neutralizing antibodies; and
(d) a negative control comprising at least one coronavirus non-neutralizing antibody.

44. The rapid detection kit of claim 43, wherein the coronavirus is coronavirus OC43, coronavirus 229E, coronavirus NL63, coronavirus HKU1, MERS-CoV, SARS-CoV, or SARS-CoV-2 (COVID-19).

45-54. (canceled)
